(19) United States
(12) Patent Application Publication
(10) Pub. No.: US 2021/0332444 A1
(43) Pub. Date: Oct. 28, 2021

(54) SARS-CoV-2 TEST KIT FOR RT-qPCR ASSAYS

(71) Applicants: Qing Sun, Richmond, CA (US); Michael Y. Sha, Castro Valley, CA (US); Hui Ren, Richmond, CA (US); Jonathan Li, Richmond, CA (US); Aiguo Zhang, San Ramon, CA (US)

(72) Inventors: Qing Sun, Richmond, CA (US); Michael Y. Sha, Castro Valley, CA (US); Hui Ren, Richmond, CA (US); Jonathan Li, Richmond, CA (US); Aiguo Zhang, San Ramon, CA (US)

(21) Appl. No.: 17/233,423

(22) Filed: Apr. 16, 2021

Related U.S. Application Data
(60) Provisional application No. 63/010,840, filed on Apr. 16, 2020.

(51) Int. Cl. C12Q 1/70

(52) U.S. Cl. CPC C12Q 1/701; C12Q 2600/158

(57) ABSTRACT

The present invention provides synthetic nucleic acid sequences comprising 10-30 nucleotides of the N1 and N2 gene regions and/or the 3′ non-coding region of the SARS-associated coronavirus Cov-2 (SARS-CoV-2) genome, and a synthetic nucleic acid sequence comprising 10-30 nucleotides of a nucleic acid sequence that is complementary to at least one of those regions. Also provided are compositions comprising the sequences, and uses of the sequences in diagnostic kits. The present invention further provides a primer and probe set for determining the presence or absence of SARS-associated coronavirus Cov-2 in a biological sample, wherein the primer set comprises at least one of the synthetic nucleic acid sequences. Also provided are a composition comprising the primer and probe set, and use of the primer and probe set in a diagnostic kit. Finally, the present invention provides kits and methods for determining the presence or absence of SARS-associated coronavirus Cov-2 (SARS-CoV-2) in a biological sample.

This application claims the priority benefit under 35 U.S.C. section 119 of U.S. Provisional Patent Application No. 63/010,840 entitled “SARS-Cov-2 Test Kit for RT-qPCR Assays” filed Apr. 16, 2020, which is in its entirety herein incorporated by reference.

### FIELD OF THE INVENTION

The present invention relates generally to the field of medicine and more specifically to infectious diseases. The invention also relates to the field of molecular biology, more particular to the detection of viral material in a biological sample.

The invention also relates generally to the field of identifying nucleic acids. More specifically the invention generally relates to diagnostic methods that may be useful for diagnosing patients infected with an agent capable of cause Severe Acute Respiratory Syndrome CoV-2 (“SARS-CoV-2”) or SAR-like symptoms.

The present invention features a method for the detection of SARS-CoV-2 in biological samples of living beings and to a kit for carrying out said method.

The present invention is also related to nucleic acid sequences that can be used in the field of virus diagnostics, more specifically the diagnosis of infections with a novel human coronavirus causing Severe Acute Respiratory Syndrome CoV-2 (SARS-CoV-2).

The present invention further relates to PCR primers and Taqman probes for detecting severe acute respiratory syndrome (SARS CoV-2) virus, a method and a kit for detecting SARS CoV-2 virus. The instant invention also relates to a quantitative real time RT-PCR method for detecting Severe Acute Respiratory Syndrome-associated virus (CoV-2 SARS-associated virus) and to oligonucleotides and kits for detecting SARS-associated CoV-2 virus.

The present invention additionally relates to nucleic acid sequences that can be used in the field of virus diagnostics, more specifically the diagnosis of infections with a novel human coronavirus causing Severe Acute Respiratory Syndrome CoV-2 virus (SARS CoV-2 virus).

### BACKGROUND OF INVENTION

Severe Acute Respiratory Syndrome (SARS) CoV-2 virus is a disease that emerged in Asia and resulted in an epidemic that had devastating health and economic effects. The disease spread rapidly from infected patient to infected patient, including numerous health care workers. Because the disease is so infectious, it is important to develop diagnostic methods to allow rapid diagnosis of this highly infectious disease. In 2019, a new Corona virus was first discovered in China named SARS-CoV-2. The new virus spread quickly from person to person. Due to intensive human travel activities, SARS-CoV-2 infected people worldwide. In March 2020 the WHO declared all criteria of a pandemic were fulfilled. In young people SARS-CoV-2 induce flu-like symptoms such as cough, sore throat, diarrhea or fever. However, severe pneumonia with fatalities also occurs in some cases especially in immunosuppressed people, old people or people with lung diseases like bronchial asthma. Up to now no suitable therapy is available. Therefore, the global community is relying on an early diagnosis with subsequent isolation of the infected people in order to slow down the further spread of SARS-CoV-2. As a result, since March 2020, many countries have introduced special measures as part of their national pandemic plans that restrict people's freedom of movement. Early diagnosis is possible using the nucleic acid amplification technique. This enables the detection of the virus directly. However, due to the exponentially increasing number of infected people, there is also a shortage of reagents with this detection method, so that not all people who are first contact persons to SARS-CoV-2 infected people or have mild flu-like symptoms can receive a diagnostic examination.

Severe acute respiratory syndrome (SARS Cov-1 and Cov-2) are relatively new potentially life threatening infectious disease of humans. After SARS Cov-1 and SARS Cov-2 were first recognized in late February 2003 in Hanoi, Vietnam, and in December 2019 in Wuhan, China respectively, the disease spread rapidly, with cases reported from all over the world on five continents over many months (World Health Organization. Severe acute respiratory syndrome (SARS I. Wkly. Epidemiol. Rec. 2003, 78:81-3; Peiris, et al. Coronavirus as a possible cause of severe acute respiratory syndrome. Lancet 2003, 361:1319-25; Lee, et al. A major outbreak of severe acute respiratory syndrome in Hong Kong. N. Eng. J. Med. 2003, 348:1986-94; Tsang, et al. A cluster of cases of severe acute respiratory syndrome in Hong Kong. N. Eng. J. Med. 2003, 348:1977-85; Poutanen, et al. Identification of severe acute respiratory syndrome in Canada. N. Eng. J. Med. 2003, 348:1995-2005; Kuiken, et al. Newly discovered coronavirus as the primary cause of severe acute respiratory syndrome. Lancet 2003, 362:263-70; World Health Organization Multicentre Collaborative Network for Severe Acute Respiratory Syndrome (SARS) Diagnosis and A multicentre collaboration to investigate the cause of severe acute respiratory syndrome. Lancet 2003, 361:1730-3). By Jul. 3, 2003, this epidemic resulted in 8,439 reported cases globally, of which 812 were fatal (Cumulative number of reported probable cases of severe acute respiratory syndrome (SARS). e-publication cited Jul. 8, 2003) and Zhu N, Zhang D, Wang W, Li X, Yang B, Song J, et al. A Novel Coronavirus from Patients with Pneumonia in China, 2019. N Engl J Med. 2020 Jan. 24.

The most common early symptoms of SARS both Cov-1 and Cov-2 include fever (a measured temperature greater than 100.4° F. (38.0° C.)), chills, headache, myalgia, dizziness, rigors, cough, sore throat, and runny nose (WHO Weekly Epidemiological Record, No. 12, Mar. 21, 2003). The SARS illness usually starts with fever, severe headache, dizziness, and myalgia. After 2 to 7 days, SARS patients generally develop a dry, nonproductive cough. In some cases, there may be rapid deterioration of conditions, with low oxygen saturation and acute respiratory distress.

The SARS-associated coronaviruses COv-1 and Cov-2 pathogens were quickly isolated, and their genomes have been sequenced by scientists in China, Canada and the United States (Ksiazek et al., A novel coronavirus associated with severe acute respiratory syndrome. N. Engl. J. Med., Apr. 10, 2003, e-pub; Drosten et al., Identification of a novel coronavirus in patients with severe acute respiratory syndrome. N. Engl. J. Med., Apr. 10, 2003, e-pub; WHO Update 31, Coronavirus never before seen in humans is the cause of SARS, Apr. 16, 2003) and Lu, R. et al. Genomic characterization and epidemiology of 2019 novel coronavirus: implications for virus origins and receptor binding. Lancet, doi:10.1016/50140-6736(20)30251-8 (2020). Rapid identification of the causal agent as a novel coronavirus (SARS-CoV-1) represents an extraordinary achievement in the history of global health and helped to contain the epidemic (World Health Organization Multicentre Collaborative Network for Severe Acute Respiratory Syndrome (SARS) Diagnosis. A multicentre collaboration to investigate the cause of severe acute respiratory syndrome. Lancet 2003 361:1730-3). Nonetheless, the epidemiology and pathogenesis of SARS for Cov-1 and Cov-2 remain poorly understood, and definitive diagnostic tests or specific treatments are not established. Since the origin of the virus and its animal reservoirs remain to be defined, the potential for recurrence is unknown. This fact underscores the importance of stablishing sensitive and efficient methods for diagnosis and surveillance.

The coronaviruses that has been implicated in SARS Cov-1 and Cov-2 represents the prototype of a new lineage of coronaviruses capable of causing outbreaks of clinically significant and frequently fatal human disease. Coronaviruses were first isolated from chicken in 1937, and from human in 1965. The coronavirus family contains approximately 15 species, which infect a broad range of animals, including humans, cats, dogs, cows, pigs, rodents, and birds (e.g., chickens, batts). The coronavirus is a single-stranded, (+)sense RNA virus. The virus enters the host cell via endocytosis, and reproduces itself in the cytoplasm; no DNA stage is involved. New virions form by budding into the Golgi apparatus, being transported to the cell surface, and secreted from host cell.

To date, there is only a limited repertoire of sensitive, specific diagnostic assays available that allow surveillance and clinical management of SARS and SARS-associated diseases both for Cov-1 and Cov-2. As specific antiviral therapies are established, early diagnosis will be increasingly important in minimizing morbidity and mortality. Immunofluorescence and enzyme-linked immunosorbent assays (ELISA) are reported to inconsistently detect antibodies to SARS-CoV-1 and Cov-2 before day 10 or 20 after the onset of symptoms, respectively (World Health Organization Multicentre Collaborative Network for Severe Acute Respiratory Syndrome (SARS) Diagnosis. A multicentre collaboration to investigate the cause of severe acute respiratory syndrome. Lancet 2003, 361:1730-3; Li G Chen X and Xu A. Profile of specific antibodies to the SARS-associated coronavirus. N. Eng. J. Med. 2003, 349:5-6). Thus, although helpful in tracking the course of infection at the population level, these serologic tools have less usefulness in detecting infection at early stages, when there may be potential to implement therapeutic interventions or measures, such as quarantine that may reduce the risk for transmission to naive persons. In contrast, polymerase chain reaction (PCR)-based assays have the potential to detect SARS Cov-1 and SARS Cov-2 and SARS-associated infections at earlier time points. However, a need exists for a sensitive, reliable, and rapid diagnostic method for detecting the presence of the SARS-associated coronavirus Cov-1 and Cov-2 in a biological sample at the earliest possible stage of infection.

Furthermore, it is known that nasopharyngeal swab (NPS) and oropharyngeal swab (OPS) samples are widely accepted as specimens for the detection of SARS-CoV-2 since the start of the COVID-19 pandemic. However, the collection procedures for NTS and OPS specimens may cause discomfort and, in some people, sneezing and coughing. The latter in turn can generate droplets or aerosol particles that place healthcare workers collecting these specimens at risk, requiring heavy use of personal protective equipment (PPE). Poor tolerability of NPS and OPS sampling can result in false-negative tests due to inadequate or poor quality of specimen collection. Recent investigations suggested that saliva is a viable and even more sensitive alternative to NPS specimens, and could also enable at-home self-administered sample collection for large-scale SARS-CoV-2 molecular testing. Other researchers also reported that SARS-CoV-2 was detected in 91.7% (n=11) of the initial saliva specimens from confirmed. COVID-19 patients. All saliva specimens (n=3) collected from patients whose NPS specimens tested negative for COVID-19 also tested negative. it is apparent that detection of SARS CoV-2 in saliva can be used as an more appealing and cost-effective alternative for the diagnosis of COVID-19. indeed, a molecular test using saliva samples was first approved for FDA under EUA on May 8, 2020.

The use of saliva specimens might decrease the risk of nosocomial transmission of COVID-19 and is ideal for situations in which NPS or OPS specimen collection may be impractical. Collecting saliva is easy and more tolerable to patients, can reduce risk of cross-infection, and can be used in settings where PPE is not readily available. It will also be useful for testing infants and young children in daycare facilities and schools.

The QuantiVirus™ SARS-CoV-2 Test is a real-time reverse transcription polymerase chain reaction (RT-qPCR) test that includes the assay controls for the qualitative detection of viral RNA from SARS-CoV-2 in NPS, OPS, saliva or sputum specimens collected from patients who are suspected of COVID-19 infection. Extracted RNA is reverse-transcribed and amplified in a single reaction. In this multiplex qPCR method, the Orflab, N, and E genes of the SARS-COV-2 genome are targeted in the RT-PCR assay. Primers and TaqMan probes designed for conserved regions of the SARS-CoV-2 virus genome allow specific amplification and detection of the viral RNA from all strains of SARS-CoV-2 from respiratory specimens. The Human RNase P gene is used as an Internal Control (IC) to monitor viral RNA extraction efficiency and assess amplifiable RNA in the samples to be tested. The test is a multiplex RT-PCR assay consisting of one reaction with primers and probes for the viral gene targets (Orflab, N and E genes) and IC in one tube, designed to increase assay throughput.

### BRIEF DESCRIPTION OF THE FIGURES

FIG. 1 shows the Amplicon Target on SARS-Cov-2 genome.

FIG. 2 is the amplification curve of 10-fold serial dilution of templates showing the threshold setting.

FIG. 3 describes high throughput workflow for SARS-COV-2 detection from sample collection to result availability within about 4 hrs.

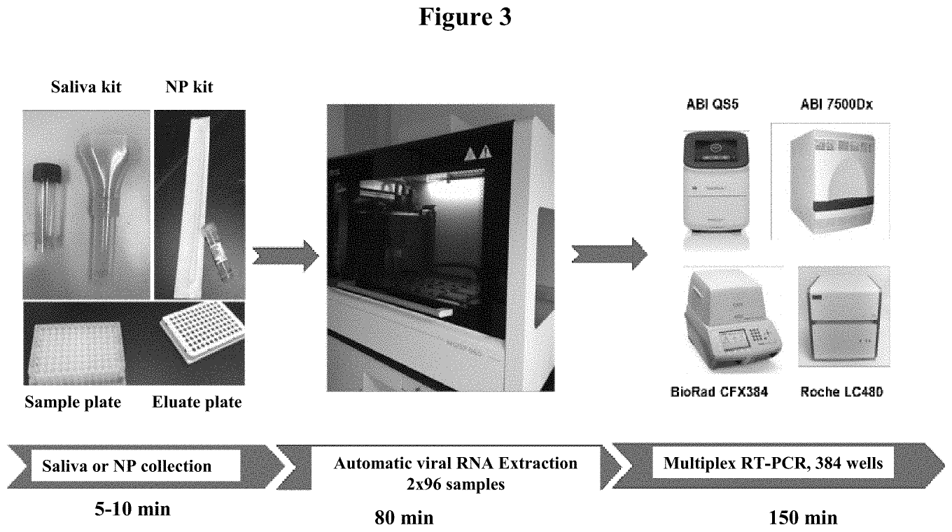

### ABBREVIATIONS USED THROUGHOUT THE SPECIFICATION

### CLIA Clinical Laboratory Improvement Amendments

### Ct Cut-Off Threshold

DNA Deoxyribonucleic acid

### EC Extraction Control

E gene Envelope small membrane protein

### EUA Emergency Use Authorization

### FDA Food and Drug Administration

### IFU Instructions for Use

mL milliliter

### LoD Limit of Detection

μL microliter

### N/A Not Available

N gene Nucleoprotein

### NPA Negative Percentage Agreement

### NPV Negative Predictive Value

### NTC No Template Control

Orfl ab gene Open Reading Frame 1 ab

### PC Positive Control

### PCR Polymerase Chain Reaction

### POS Positive

### PPA Positive Percentage Agreement

### PPV Positive Predictive Value

RNA Ribonucleic acid

### RP RNase P

### SAE Serious Adverse Event

### VTM DiaCarta QuantiVirus™ Viral Transport Medium

UTM HealthLink Transport medium

### SUMMARY OF THE INVENTION

The present invention provides SARS-CoV-2-specific primers and Taqman probes.

The present invention also provides a method for specifically detecting SARS-CoV-2 using the primers and probes.

The present invention also provides a SARS-CoV-2 detection kit including the primers and Taqman probes.

The instant invention also relates to an efficient, sensitive and reliable quantitative real time RT-PCR method for detecting Severe Acute Respiratory Syndrome Coronavirus (SARS CoV-2) and to oligonucleotides and kits for detecting SARS CoV-2.

The QuantiVirus™ SARS-CoV-2 Test kit of the invention is a real-time RT-PCR test intended for the qualitative detection of nucleic acid from the SARS-CoV-2 in nasopharyngeal swabs, oropharyngeal swabs and sputum from individuals suspected of COVID-19. Testing is limited to laboratories—certified under the Clinical Laboratory Improvement Amendments of 1988 (CLIA), 42 U.S.C. § 263a, to perform high complexity tests, or by similarly qualified non-U.S. laboratories.

Results are for the identification of SARS-CoV-2 RNA. The SARS-CoV-2 RNA is generally detectable in sputum and upper respiratory specimens during the acute phase of infection. Positive results are indicative of active infection. Laboratories within the United States and its territories are required to report all positive results to the appropriate public health authorities.

According to another aspect of the present invention, there is provided a method for detecting SARS-CoV-2, which includes amplifying a nucleic acid sample obtained from an individual by PCR using the primers and probes of the invention.

According to yet another aspect of the present invention, there is provided a SARS-CoV-2 detection kit including the primers and probes.

The invention is also a method for detecting SARS-associated corona virus Cov-2 (SARS-CoV-2) by contacting a biological sample with a set of primers and a probe, incubating under conditions allowing amplification of nucleic acid using said primers, and determining binding of said probe to amplified nucleic acid, wherein detecting binding of said probe to amplified nucleic acid indicates the presence of SARS-associated virus, wherein the primers are selected from the group consisting of the following primer sets: (a) a primer set comprising a primer consisting of WHnCoVF2 SEQ ID NO: 1 GTTCCAATTAACACCAATAGCA and a primer WHnCoVR2a SEQ ID NO: 2 ATTCGTCTGGTAGCTCTTC; (b) a primer set comprising a primer consisting of WHnCoVF3 SEQ ID NO: 4 GCAAATTCTATGGTGGTTGG and a primer consisting of WHnCoVR3 SEQ ID NO: 5 GCATGGCTCTATCACATTTAG; (c) a primer set comprising a primer consisting of WHnCoVF4 SEQ ID NO: 7 GCTTCGATTGTGTGCGTAC and a primer consisting of WHnCoVR4 SEQ ID NO: 8 GACCAGAAGATCAGGAACTCTA; and (d) a primer set comprising a primer consisting of RP-FSEQ ID NO: 10 AGATTTGGACCT GCGAGCG and a primer consisting of RP-R SEQ ID NO: 11 GAGCGGCTGTCTCCACAAG T; and wherein the probe is selected from the group consisting of WHnCoVPr2 (Probe) SEQ ID NO: 3 TCCAGATGACCAAATTGGCTAC; WHnCoVPr3 (Probe) SEQ ID NO: 6 ACTGTTTATAG TGATGTAGAAAACCCTCA; WHnCoVPr4(Probe) SEQ ID NO: 9 CTGCAATATTGTTAAC GTGAGTCTTGT; and RP-P (Probe) SEQ ID NO: 12 TTCTGACCTGAAGGCTCTGCG CG; and wherein the probe is labeled with two dyes, one dye of which is a fluorescent reporter dye, and one dye of which is a quencher dye, and wherein at least one dye is a fluorescent dye; and the SARS virus is detected by detection of real time fluorescence, if amplification of virus specific sequence occurs.

The invention also provides a kit for detecting SARS-associated corona virus Cov-2 (SARS-CoV-2) in a biological sample comprising a PCR primer set selected from the group consisting of the following primer sets: (a) a primer set comprising a primer consisting of WHnCoVF2 SEQ ID NO: 1 GTTCCAATTAACA CCAATAGCA and a primer WHnCoVR2a SEQ ID NO: 2 ATTCGTCTGGTAGCTCTTC (b) a primer set comprising a primer consisting of WHnCoVF3 SEQ ID NO: 4 GCAAATTCTATGGTGGTTGG and a primer consisting of WHnCoVR3 SEQ ID NO: 5 GCATGGCTCTATCACATTTAG (c) a primer set comprising a primer consisting of WHnCoVF4 SEQ ID NO: 7 GCTTCGATTGTGTGCGTAC and a primer consisting of WHnCoVR4 SEQ ID NO: 8 GACCAGAAGATCAGGAACTCTA; and (d) primer set comprising a primer consisting of RP-FSEQ ID NO: 10 AGATTTGGACCTGCGAG CG and a primer consisting of RP-R SEQ ID NO: 11 GAGCGGCTGTCTCCACAAGT; wherein the primer set specifically amplifies a target region of Severe Acute Respiratory syndrome corona virus CoV-2 (SARS-CoV-2) in a polymerase chain reaction (PCR).

### DETAILED DESCRIPTION OF THE INVENTION

Whereas conventional virus diagnosis has been based predominantly on the detection of viral antigens or specific antibodies thereto, in recent years attention has shifted towards methods for the direct and rapid detection of the genome of viruses or nucleic acid sequences derived thereof, both RNA and DNA. In this respect, the very short time-to-result is a crucial factor to opt for nucleic acid detection. These methods are usually based on nucleic acid hybridization. Nucleic acid hybridization is based on the ability of two strands of nucleic acid containing complementary sequences to anneal to each other under the appropriate conditions, thus forming a double stranded structure. When the complementary strand is labeled, the label can be detected and is indicative for the presence of the target sequence. Especially in combination with methods for the amplification of nucleic acid sequences these methods have become an important tool in viral diagnosis.

Nucleic acid amplification techniques are especially useful as an additional technique in cases where serological methods give doubtful results or in cases where there may be a considerable time period between infection and the development of antibodies to the virus.

The choice of the oligonucleotides to be used as primers and probes in the amplification and detection of nucleic acid sequences is critical for the sensitivity and specificity of the assay. The sequence to be amplified is usually only present in a sample (for example a blood sample obtained from a patient suspected of having a viral infection) in minute amounts. The primers should be sufficiently complementary to the target sequence to allow efficient amplification of the viral nucleic acid present in the sample. If the primers do not anneal properly (due to mispairing of the bases on the nucleotides in both strands) to the target sequence, amplification is seriously hampered. This will affect the sensitivity of the assay and may result in false negative test results. Due to the heterogeneity of viral genomes false negative test results may be obtained if the primers and probes are capable of recognizing sequences present in only part of the variants of the virus.

The present invention provides a PCR primer set useful for detecting SARS-CoV-2 selected from the group consisting of the following primer sets: (a) a primer set comprising a primer consisting of WHnCoVF2 SEQ ID NO: 1 GTTCCAATTAACACCAATAGCA and a primer WHnCoVR2a SEQ ID NO: 2 ATTCGTCTGGTAGCTCTTC; (b) a primer set comprising a primer consisting of WHnCoVF3 SEQ ID NO: 4 GCAAATTCTATGGTGGTTGG and a primer consisting of WHnCoVR3 SEQ ID NO: 5 GCATGGCTCTATCACATTTAG; (c) a primer set comprising a primer consisting of WHnCoVF4 SEQ ID NO: 7 GCTTCGATTGTGTGCGTAC and a primer consisting of WHnCoVR4 SEQ ID NO: 8 GACCAGAAGATCAGGAACTCTA; and (d) a primer set comprising a primer consisting of RP-FSEQ ID NO: 10 AGATTTGGACC TGCGAGCG and a primer consisting of RP-R SEQ ID NO: 11 GAGCGGCTGTCTCCACAAG T; wherein the primer set specifically amplifies a target region of Severe Acute Respiratory syndrome corona virus CoV-2 (SARS-CoV-2) in a polymerase chain reaction (PCR).

The invention further provides oligonucleotide, for use as a probe to detect the amplified nucleic acid sequence resulting in the amplification of a target sequence located within the genome of SARS Coronavirus-2, said amplification being based on pair of oligonucleotides according to claim 1, said probe being selected from the group consisting of WHnCoVPr2 (Probe) SEQ ID NO: 3 TCCAGATGACCAAATTGGCTAC; WHnCoVPr3(Probe) SEQ ID NO: 6 ACTGTTTATA GTGATGTAGAAAACCCTCA; WHnCoVPr4(Probe) SEQ ID NO: 9 CTGCAATATTGTTAA CGTGAGTCTTGT; and RP-P (Probe) SEQ ID NO: 12 TTCTGACCTGAAGGCTCTGC GCG.

The invention relates to a method for detecting Severe Acute Respiratory Syndrome-associated virus CoV-2 (SARS-CoV-2), wherein a real time RT-PCR reaction is performed using a biological sample. Based on the sequence data, an efficient, sensitive and reliable quantitative real time RT PCR method was developed. In one embodiment, the primers are selected from the group consisting of the following primer sets: (a) a primer set comprising a primer consisting of WHnCoVF2 SEQ ID NO: 1 GTTCCAATTAACACCAATAGCA and a primer WHnCoVR2a SEQ ID NO: 2 ATTCGTCTGGTAGCTCTTC; (b) a primer set comprising a primer consisting of WHnCoVF3 SEQ ID NO: 4 GCAAATTCTATGGTGGTTGG and a primer consisting of WHnCoVR3 SEQ ID NO: 5 GCATGGCTCTATCACATTTAG; (c) a primer set comprising a primer consisting of WHnCoVF4 SEQ ID NO: 7 GCTTCGATTGTGTGCGTAC and a primer consisting of WHnCoVR4 SEQ ID NO: 8 GACCAGAAGATCAGGAACTCTA; and (d) a primer set comprising a primer consisting of RP-FSEQ ID NO: 10 AGATTTGGACC TGCGAGCG and a primer consisting of RP-R SEQ ID NO: 11 GAGCGGCTGTCTCCACAAG T; and wherein the probe is selected from the group consisting of WHnCoVPr2 (Probe) SEQ ID NO: 3 TCCAGATGACCAAATTGGCTAC; WHnCoVPr3(Probe) SEQ ID NO: 6 ACTGTTTA TAGTGATGTAGAAAACCCTCA; WHnCoVPr4(Probe) SEQ ID NO: 9 CTGCAATATTGTT AACGTGAGTCTTGT; and RP-P (Probe) SEQ ID NO: 12 TTCTGACCTGAAGGCTCTGC GCG; and wherein the probe is labeled with two dyes, one dye of which is a fluorescent reporter dye, and one dye of which is a quencher dye, and wherein at least one dye is a fluorescent dye; and the SARS virus is detected by detection of real time fluorescence, if amplification of virus specific sequence occurs.

Useful fluorescent dyes and qunechers for using with the probes are listed in Table below.

| | Max. EX | Max. EM | |
|---|---|---|---|
| Dye | (nm) | (nm) | Compatible Quencher |
| 6-FAM ™ | 494 | 515 | BHQ-1, DABCYL |
| Fluorescein | 495 | 520 | BHQ-1, DABCYL |
| JOE ™ | 520 | 548 | BHQ-1, DABCYL |
| TET | 521 | 536 | BHQ-1, DABCYL |
| Cal Fluor ® Gold 5401 | 522 | 541 | BHQ-1 |
| HEX | 535 | 555 | BHQ-1, DABCYL |
| Cal Fluor Orange 5602 | 540 | 561 | BHQ-1 |
| TAMRA ™ | 555 | 576 | BHQ-2 |
| Cyanine 3 | 550 | 570 | BHQ-2, DABCYL |
| Quasar ® 5703 | 548 | 566 | BHQ-2 |
| ROX ™ | 573 | 602 | BHQ-2, DABCYL |
| Texas Red ® | 583 | 603 | BHQ-2, DABCYL |
| Cyanine 5 | 651 | 674 | BHQ-3, DABCYL |
| Quasar 6705 | 647 | 667 | BHQ-3 |
| Cyanine 5.5 | 675 | 694 | BHQ-3, DABCYL |

The invention also provides a kit for detecting SARS-associated corona virus Cov-2 (SARS-CoV-2) in a biological sample comprising a PCR primer set selected from the group consisting of the following primer sets: (a) a primer set comprising a primer consisting of WHnCoVF2 SEQ ID NO: 1 GTTCCAATTAACA CCAATAGCA and a primer WHnCoVR2a SEQ ID NO: 2 ATTCGTCTGGTAGCTCTTC (b) a primer set comprising a primer consisting of WHnCoVF3 SEQ ID NO: 4 GCAAATTCTATGGTGGTTGG and a primer consisting of WHnCoVR3 SEQ ID NO: 5 GCATGGCTCTATCACATTTAG; (c) a primer set comprising a primer consisting of WHnCoVF4 SEQ ID NO: 7 GCTTCGATTGTGTGCGTAC and a primer consisting of WHnCoVR4 SEQ ID NO: 8 GACCAGAAGATCAGGAACTCTA; and (d) primer set comprising a primer consisting of RP-FSEQ ID NO: 10 AGATTTGGACCTGCGAG CG and a primer consisting of RP-R SEQ ID NO: 11 GAGCGGCTGTCTCCACAAGT; wherein the primer set specifically amplifies a target region of Severe Acute Respiratory syndrome corona virus CoV-2 (BARS-CoV-2) in a polymerase chain reaction (PCR).

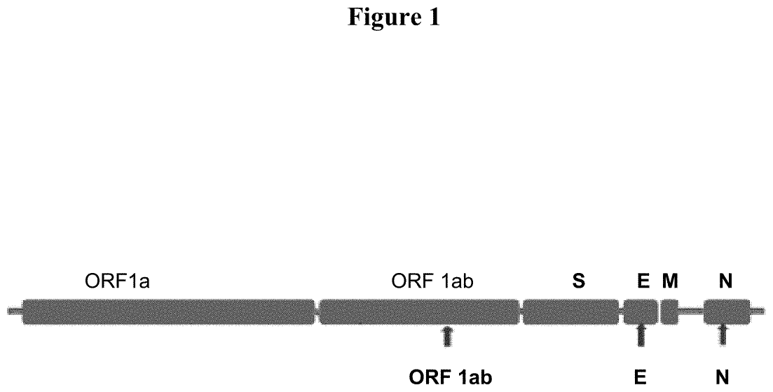

The primers and probes of the present invention can specifically detect SARS-CoV-2 without reacting with other coronaviruses. That is, when qPCR is performed using the primer and probe set of the present invention, PCR products are obtained from individuals infected with SARS-CoV-2 but no PCR products are obtained from individuals infected with other coronaviruses. The PCR primers and probes for SARS-CoV-2 detection of the present invention are selected from a non-structural region and a structural region among the genome sequence of SARS-CoV-2. SARS-CoV-2 regions including target nucleotide sequences for the primers and probes according to the present invention are illustrated in FIG. 1.

The present invention also provides a method for detecting SARS-CoV-2, which comprises amplifying a nucleic acid sample obtained from an individual by qPCR using the primers and probes for SARS-CoV-2 detection.

As used herein, the term “PCR” is well known in the pertinent art. Generally, PCR includes the steps of: (a) obtaining a crude extract containing target cDNA or DNA molecules from a sample; (b) adding an aqueous solution including an enzyme, a buffer, dNTPs, and oligonucleotide primers to the crude extract; (c) amplifying the target DNA molecules by two- or three-step thermal cycling (e.g., 90-96° C., 72° C., and 37-55° C.) of the resultant mixture; and (d) detecting amplified DNAs. In the present invention, the PCR may be performed in a polypropylene tube, a 96-well plate, or a silicon-based micro PCR chip.

When the PCR is performed on a silicon-based micro PCR chip, a two-step thermal cycling as well as a three-step thermal cycling can be used. A time required for the PCR on the silicon-based micro PCR chip can be as short as 30 minutes or less. For example, the silicon-based micro PCR chip includes a silicon wafer, a surface of which is formed with a PCR chamber made by silicon lithography and the other surface is formed with a heater for heating the PCR chamber; and a glass wafer having an inlet and an outlet.

In the present invention, the PCR may be performed using 0.2-1 μM of each primer and 0.01 pg to 1 μg of a template DNA.

In the present invention, the PCR may be performed in three-step thermal cycling conditions of denaturation at 86-97° C. for 1-30 seconds, annealing at 50-70°. C. for 1-30 seconds, and extension at 60-72° C. for 1-30 seconds, or in two-step thermal cycling conditions of denaturation at 86-97° C. for 1-30 seconds and annealing and extension at 50-70° C. for 5-30 seconds.

The present invention also provides a SARS-CoV-2 detection kit including the primers and probes for SARS-CoV-2 detection.

The SARS-CoV-2 detection kit of the present invention may include the primers, probe, a PCR solution, a buffer, an enzyme, and the like.

In a preferred embodiment, Applicants use real-time polymerase chain reaction (real-time PCR), also known as quantitative polymerase chain reaction (qPCR), is a laboratory technique of molecular biology based on the polymerase chain reaction (PCR). It monitors the amplification of a targeted DNA molecule during the PCR (i.e., in real time), not at its end, as in conventional PCR. Real-time PCR can be used quantitatively (quantitative real-time PCR) and semi-quantitatively (i.e., above/below a certain amount of DNA molecules) (semi-quantitative real-time PCR).

Two common methods for the detection of PCR products in real-time PCR are (1) non-specific fluorescent dyes that intercalate with any double-stranded DNA and (2) sequence-specific DNA probes consisting of oligonucleotides that are labelled with a fluorescent reporter, which permits detection only after hybridization of the probe with its complementary sequence.

As is commonly known, real-time PCR is carried out in a thermal cycler with the capacity to illuminate each sample with a beam of light of at least one specified wavelength and detect the fluorescence emitted by the excited fluorophore. The thermal cycler is also able to rapidly heat and chill samples, thereby taking advantage of the physicochemical properties of the nucleic acids and DNA polymerase.

The PCR process generally consists of a series of temperature changes that are repeated 25-50 times. These cycles normally consist of three stages: the first, at around 95° C., allows the separation of the nucleic acid's double chain; the second, at a temperature of around 50-60° C., allows the binding of the primers with the DNA template; the third, at between 68-72° C., facilitates the polymerization carried out by the DNA polymerase. Due to the small size of the fragments the last step is usually omitted in this type of PCR as the enzyme is able to increase their number during the change between the alignment stage and the denaturing stage. In addition, in four step PCR the fluorescence is measured during short temperature phase lasting only a few seconds in each cycle, with a temperature of, for example, 80° C., in order to reduce the signal caused by the presence of primer dimers when a non-specific dye is used. The temperatures and the timings used for each cycle depend on a wide variety of parameters, such as: the enzyme used to synthesize the DNA, the concentration of divalent ions and deoxyribonucleotides (dNTPs) in the reaction and the bonding temperature of the primers.

In another embodiment Applicant has found that saliva sampling is an adequate alternative to NPS and OPS sampling and can be used for COVID-19 testing using the QuantiVirus SARS-CoV-2 test of the invention. The use of saliva specimens might decrease the risk of nosocomial transmission of COVID-19 and is ideal for situations in which NPS or OPS specimen collection may be impractical. Collecting saliva is easy and more tolerable to patients, can reduce risk of cross-infection, and can be used in settings where PPE is not readily available. It will also be useful for testing infants and young children in daycare facilities and schools.

The SARS-CoV-2 saliva test is a real-time reverse transcription polymerase chain reaction (RT-qPCR) test that includes the assay controls for the qualitative detection of viral RNA from SARS-CoV-2 in NPS, OPS, saliva or sputum specimens collected from patients who are suspected of COVID-19 infection. Extracted RNA is reverse-transcribed and amplified in a single reaction. In this multiplex qPCR method, the Orflab, N, and E genes of the SARS-CoV-2 genome are targeted in the RT-PCR assay (See FIG. 1). Primers and TaqMan probes designed for conserved regions of the SARS-CoV-2 virus genome allow specific amplification and detection of the viral RNA from all variants of SARS-CoV-2 from respiratory specimens. The Human RNase P gene is used as an Internal Control (IC) to monitor viral RNA extraction efficiency and assess amplifiable RNA in the samples to be tested. The test is a multiplex RT-PCR assay consisting of one reaction with primers and probes for the viral gene targets (Orflab, N and E genes) and IC in one tube, designed to increase assay throughput.

Hereinafter, the present invention will be described more specifically by Examples. However, the following Examples are provided only for illustrations and thus the present invention is not limited to or by them.

### Example I

### Assay Summary

The QuantiVirus™ SARS-CoV-2 Test kit of the invention is a real-time reverse transcription polymerase chain reaction (rRT-PCR) test. The SARS-CoV-2 primer and probe set(s) is designed to detect RNA from the SARS-CoV-2 in respiratory specimens and saliva from patients as recommended for testing by public health authority guidelines.

Extracted RNA from clinical samples is reverse-transcribed and amplified in a single reaction. Three genes of the SARS-CoV-2 (FIG. 1) including N, Orflab and E genes are targeted in the qRT-PCR assay. Primers and Taqman probes are designed in the conserved region of the SARS-CoV-2 virus specific genome region to allow specific amplification and detection of viral RNA from all strains of SARS-CoV-2 from respiratory specimens. The human Rnase P gene is used as internal and extraction control to monitor viral RNA extraction efficiency and assesses amplifiable RNA/DNA in the samples to be tested. The assay is a multiplex RT-PCR assay consisting of one reaction with primers and probes for the viral targets (Orflab, N and E genes) and internal control in one tube thus with increased assay throughput and ease of use and other advantages as a multiplex assay.

FIG. 1 shows the Amplicon Target on SARS-Cov-2 genome. E: envelope protein gene; M: membrane protein gene; N: nucleocapsid protein gene; ORF: open reading frame; RdRp: RNA-dependent RNA polymerase gene; S: spike protein gene. Red arrow indicates that DiaCarta detection kit's Amplicon Target on SARS-Cov-2 genome.

### Terminology, Kit Components, Instruments, and Handling Precautions

### Terminology

a. Positive Control (PC)

A positive control is a mix of synthetic DNA templates for each target of sequences for N, E and Orflab genes of the SARS-CoV2 genome at a concentration of 1×10 4 copies/μL. Positive controls must show the appropriate values in both target (FAM and HEX) channels for the run to be valid. Positive control monitors the function of each assay component.

b. No Template Control (NTC)

Nuclease free water is used in place of template. No amplification should be observed in all channels, assuring the absence of contamination during assay set-up.

c. Extraction/Internal Control Gene Target:

Human Rnase P gene is used to monitor RNA extraction for each sample. It also serves. to monitor the assay (both reverse transcriptase and qPCR). A positive Rnase P assay demonstrates successful RNA extraction and assay.

TABLE 1
RNA Extraction and Assay Control

| Control | Used to monitor | Assays |
|---|---|---|
| Positive Control | RT-PCR reaction | Three genes assay |
| No Template Control | Cross contamination for | Three genes assay |
| | assay procedure | |
| Extraction/Internal | RNA extraction, reverse | Rnase P gene assay |
| Control Gene Target | transcription and qPCR | |

Kit Components—The kit components are listed in Table 2.

TABLE 2
Package contents - 24 reactions and 48 reactions kit

| | | | Label Volume | Label Volume | |
|---|---|---|---|---|---|
| Name of | | Pack | for each Vial | for each Vial | Storage |
| Component | Description | Size | (48 Reactions) | (24 Reactions) | Temp. |
| 5x Primer/ | Primer/probe Mix A (N gene): | 4 vials | 96 μL | 48 μL | −25° C. to |
| probe mix | N gene primers and probe | | | | −15° C. |
| | Primer/probe Mix B (Orf1ab gene): | | | | |
| | Orf1ab gene primers and probe | | | | |
| | Primer/probe Mix C (E gene) | | | | |
| | E gene primers and probe | | | | |
| | Primer/probe Mix D (Human | | | | |
| | Rnase P gene) | | | | |
| | Human Rnase P gene primers | | | | |
| | and probe | | | | |
| One step | TaqPath 1-step Multiplex | 1 vial | 480 μL | 240 μL | −25° C. to |
| qRT-PCR | Master mix | | | | −15° C. |
| Master Mix | | | | | |
| Positive | Synthetic DNA templates | 1 vial | 40 μL | 24 μL | −25° C. to |
| Controls | (Positive control, PC) for N, | | | | −15° C. |
| | Orf1ab and E genes | | | | |
| No Template | Nuclease-Free Water | 1 vial | 960 μL | 480 μL | −25° C. to |
| Control | | | | | −15° C. |

### Primer and Probe Sequences

| Target | Sequence | | 5′ | 3′ |
|---|---|---|---|---|
| Gene | Name | Sequence | modify | modify |
| N Gene | WHnCoVF2 | SEQ ID NO: 1 GTTCCAATTAACACCAATAGCA | FAM | BHQ-1 |
| | WHnCoVR2a | SEQ ID NO: 2 ATTCGTCTGGTAGCTCTTC | | nova |
| | WHnCoVPr2 | SEQ ID NO: 3 TCCAGATGACCAAATTGGCTAC | | |
| | (Probe) | | | |
| ORF1ab | WHnCoVF3 | SEQ ID NO: 4 GCAAATTCTATGGTGGTTGG | FAM | BHQ-1 |
| | WHnCoVR3 | SEQ ID NO: 5 GCATGGCTCTATCACATTTAG | | nova |
| | WHnCoVPr3 | SEQ ID NO: 6 ACTGTTTATAGTGATGTAGAAA | | |
| | (Probe) | ACCCTCA | | |
| E Gene | WHnCoVF4 | SEQ ID NO: 7 GCTTCGATTGTGTGCGTAC | FAM | BHQ-1 |
| | WHnCoVR4 | SEQ ID NO: 8 GACCAGAAGATCAGGAACTCTA | | plus |
| | WHnCoVPr4 | SEQ ID NO: 9 CTGCAATATTGTTAACGTGAGTCTTGT | | |
| | (Probe) | | | |
| Human | RP-F | SEQ ID NO: 10 AGATTTGGACCTGCGAGCG | HEX | BHQ-1 |
| Rnase P | RP-R | SEQ ID NO: 11 GAGCGGCTGTCTCCACAAGT | | nova |
| Gene | RP-P(Probe) | SEQ ID NO: 12 TTCTGACCTGAAGGCTCTGCGCG | | |

QuantiVirus™ SARS-CoV-2 Test kit Synthetic target were synthesized from IDT:

a. WHnCoV gBlock1 for N 1 gene
SEQ ID NO: 13
GACAAGGAACTGATTACAAACATTGGCCGCAAATTGCACAATTTGCCCCC
AGCGCTTCAGCGTTCTTCGGAATGTCGCGCA

[symbol]

GGGAACGTGGTTGACCTACACAGGTGCCATCAAATTGGATGACAAAG
ATCCAAATTTCAAAGATCAAGTCATTTTGCTGAATAAGCATATTGACGCA
b. WHnCoV gBlock2 for N 2 gene
SEQ ID NO: 14
ACCGCTCTCACTCAACATGGCAAGGAAGACCTTAAATTCCCTCGAGGACA
AGGCGTTCCAATTAACACCAATAGCAG [symbol] T
ACCGAAGAGCTACCAGACGAATTCGTGGTGGTGACGGTAAAATGAAAGAT
CTCAGTCCAAGATGGTATTTCTACTACCTAGGAACTGGGCCAGAAGCTGG
c. WHnCoV gBlock3 Orf1ab
SEQ ID NO: 15
ACCGTAGCTGGTGTCTCTATCTGTAGTACTATGACCAATAGACAGTTTCA
TCAAAAATTATTGAAATCAATAGCCGCCACTAGAGGAGCTACTGTAGTAA
TTGGAACAAGCAAATTCTATGGTGGTTGGCACAACATGTTAAAA

[symbol]

[symbol]

CCTTATGGGTTGGGATTATCCTAAAT
GTGATAGAGCCATGCCTAACATGCTTAGAATTATGGCCTCACTTGTTCTT
GCTCGCAAACATACAACGTGT
d. WHnCoV gBlock4 for E gene
SEQ ID NO: 16
ATGTACTCATTCGTTTCGGAAGAGACAGGTACGTTAATAGTTAATAGCGT
ACTTCTTTTTCTTGCTTTCGTGGTATTCTTGCTAGTTACACTAGCCATCC
TTACTGCGCTTCGATTGTGTGCGTACTG

[symbol]

AAAACCTTCTTTTTACGTTTACTCTCGTGTTAAAAATCTGAATTC
TTCTAGAGTTCCTGATCTTCTGGTCTAAACGAACTAAATATTATATTAGT
TTTTCTGTTTGGAACTTTAATTTTAGCCATGGCAGATTCCAACGGTACTA
TTACCGTTGAAGAGCTTAAAAAGCTCCTTGAACAAT

Primers are underlined probes are in bolded and italic font Materials Required

A. Reagents for Viral RNA Isolation

RNA quality and quantity are critical for the test accuracy. The following commercial* kits are recommended for the isolation of viral RNA from clinical samples.

### 1. Qiagen QIAamp Viral RNA Mini Kit, Cat No./ID: 52904

2. Thermo Fisher PureLink viral RNA/DNA mini kit cat #122800500
*Follow the vendor's Instructions For Use (IFU)/Product Insert

### B. Consumables

Nuclease-free, low-binding microcentrifuge tubes
   Nuclease-free pipet tips with aerosol barriers

### C. Other Reagents

Molecular grade DNase/RNase free water

### D. Equipment

qPCR instrument (equivalent to ABI 7500 Dx)

Dedicated pipettes* (adjustable, 10-100 μL, 100-200 μL, 1000 μL) for sample preparation
Dedicated pipettes* (adjustable, 1-20 μL, 10-100 μL, 100-200 μL, 1000 μL) for PCR Master Mix preparation
Dedicated pipettes* (adjustable, 1-20 μL, 10-100 μL) for dispensing of template RNA/DNA
12-channel multichannel pipettor (P-10) for transferring reactions to PCR plates.
Microcentrifuge
Benchtop centrifuge* with rotor for 1.5 mL tubes
Benchtop mini centrifuge with rotor for PCR strips
Benchtop plate centrifuge
Vortexer
96-well PCR plate/384-well PCR plate
Clear PCR plate sealer
Reagent reservoir (holding 25 ml liquid or more)
Spectrophotometer
   Note: * Prior to use ensure that instruments and equipment have been maintained and calibrated according to the manufacturer's recommendations.

### Instruments

The assays have been developed on the instruments shown in the Table 3 below. Important Note: To get reliable results, assay must be performed using the calibrated/validated instruments.

TABLE 3
Recommended List of instruments for this kit

| Company | Model |
|---|---|
| BioRad | CFX384 |
| Thermo Fisher (ABI) | QuantStudio 5 |
| Thermo Fisher (ABI) | 7500 Fast Dx |

### Handling and Storage

This kit is shipped on dry ice. If any component of the kit is not frozen on arrival, the outer packaging has been opened during transit, or the shipment does not contain a packaging note or the reagents, please contact DiaCarta or the local distributors as soon as possible.

The kit should be stored at −20° C. immediately upon receipt at −15° C. to −25*C in a constant-temperature freezer and must be protected from light. When stored under the specified storage conditions, the kit is stable until the stated expiration date. It is recommended to store the PCR reagents (Box 1 and 2) in a pre-amplification area and the controls (Box 3) in a postamplification (DNA template-handling) area. The kit can undergo up to 6 freeze-thaw cycles without affecting performance.

All reagents must be thawed at ambient temperature for a minimum of 30 minutes before use. Do not exceed 2 hours at ambient temperature. The primer and probe mixes contain fluorophore labeled probes and should be protected from light.

Attention should be paid to expiration dates and storage conditions printed in the box and labels of all components. Do not use expired or incorrectly stored components.

### General Considerations

Effective use of qPCR tests requires good laboratory practices, including maintenance of equipment that is dedicated to molecular biology. Use nuclease-free lab ware (pipettes, pipette tips, reaction vials) and wear gloves when performing the assay. Use aerosol-resistant pipette tips for all pipetting steps to avoid cross contamination of the samples and reagents.

Prepare the assay mixes in designated pre-amplification areas using only equipment dedicated to this application. Add template RNA/DNA in a separate area (preferably a separate room). Use extreme caution to prevent RNase and DNase contamination that could result in degradation of the template RNA/DNA, or PCR carryover contamination, which could result in a false positive signal.

Reagents supplied are formulated specifically for use with this kit. Make no substitutions in order to ensure optimal performance of the kit. Further dilution of the reagents or alteration of incubation times and temperatures may result in erroneous or discordant data.

### Warnings and Precautions

Use extreme caution to prevent contamination of PCR reactions with the positive and negative controls provided.
Minimize exposure of the 4×PCR Master Mix to room temperature for optimal amplification.
Avoid over exposure of the primer-probe mixes to light for optimal fluorescent signal. Use of non-recommended reagent volumes may result in a loss of performance and may also decrease the reliability of the test results.
Use of non-recommended volumes and concentrations of the RNA/DNA sample may result in a loss of performance and may also decrease the reliability of the test results.
Use of non-recommended consumables with instruments may adversely affect test results.
Do not re-use any remaining reagents after PCR amplification is completed.
Additional validation testing by user may be necessary when using non-recommended instruments.
Perform all experiments under proper sterile conditions using aseptic techniques.
Perform all procedures using universal precautions.
Wear personal protective apparel, including disposable gloves, throughout the assay procedure.
Do not eat, drink, smoke, or apply cosmetics in areas where reagents or specimens are handled.
Dispose of hazardous or biologically contaminated materials according to the practices of your institution.
Discard all materials in a safe and acceptable manner, in compliance with all legal requirements.
Dissolve reagents completely, then mix thoroughly by pipetting up and down several times or vertexing if needed.
If exposure to skin or mucous membranes occurs, immediately wash the area with large amounts of water. Seek medical advice immediately.
Do not use components beyond the expiration the date printed on the kit boxes.
Do not mix reagents from different lots.
Return all components to the appropriate storage condition after preparing the working reagents.
Do not interchange vial or bottle caps, as cross-contamination may occur.
Keep all the materials on ice when in use.
Do not leave components out at room temperature for more than 2 hours.
Reagents supplied are formulated specifically for use with this kit. Make no substitutions in order to ensure optimal performance of the kit. Further dilution of the reagents or alteration of incubation time and temperature may result in erroneous or discordant data.

The product contains no substances which at their given concentration, are considered to be hazardous to health or environment.

### HMIS

### Health 0

### Flammability 0

### Reactivity 0

### Instructions for Use

### Viral RNA Isolation

Please refer to the IFU (Instructions for Use) of the chosen commercial extraction kit for usage details. Several methods exist for RNA isolation. For consistency, we recommend using the following commercial kit:
QIAamp® Viral RNA Mini Kit (Qiagen Cat. 52904/52906)
PureLink™ Viral RNA/DNA Mini Kit (Invitrogen Cat. 12280050)

Follow the RNA isolation procedure according to manufacturer's protocol. Up to 5.5 μL of the extracted RNA can be used in 1 reaction. After RNA isolation, use spectrophotometer to check the RNA concentration, make sure the A260/A280 value is −2.0. Use extreme precautions to handle RNA samples to prevent RNA degradation caused by RNases, ware gloves all the time during the whole process, and preferably in an area specific for RNA work, use DEPC treated water and containers, etc. Store extracted RNA at −80° C. prior to use.

### Preparation of Reagents and Assay Mixes

Thaw all primer and probe mixes, Positive Control, Nuclease-Free Water and 4×qRT-PCR Master Mix provided. Thaw all reaction mixes at room temperature for a minimum of 30 minutes. Vortex all components except the PCR Master Mix and Primer and probe Mix for 5 seconds and perform a quick spin. The qRT-PCR Master Mix and Primer/probe mix should be mixed gently by inverting the tube a few times.

Prior to use, ensure that any precipitate in the qRT-PCR Master Mix is re-suspended by pipetting up and down multiple times. Do not leave kit components at room temperature for more than 2 hours. The PCR reactions are set up in a total volume of 10 μL/reaction. Table 4 shows the component volumes for each 10 ul reaction.

TABLE 4
Assay components and reaction volume

| Components | Volume/Reaction |
|---|---|
| 4X qRT - PCR Master | 2.5 μL |
| Primer and Probe Mix | 2 μL |
| RNA sample or | Sample - 5.5 μL |
| Controls* | Controls - add 2 μL of controls and add 3.5 μL |
| | of nuclease free water to make 5.5 μL volume |
| Total Volume | 10 μL |

For accuracy, 4×PCR Master Mix, primers and probes should be pre-mixed into assay mixes as described in Table 5 below.

### Preparation of Assay Mixes

Assay mixes should be prepared just prior to use. Label a micro centrifuge tube (not provided) for each reaction mix, as shown in Table 5. For each control and virus detection reaction, prepare sufficient working assay mixes for the RNA samples, one Positive Control, one Nuclease-Free Water for No-Template Control (NTC), according to the volumes in Table 4. Include reagents for 1 extra sample to allow sufficient overage for the PCR set-up. The assay mixes contain all of the components needed for PCR except the sample.

TABLE 5
Preparation of assay mixes

| | Volume of 4X | Volume of Primer |
|---|---|---|
| | PCR Master Mix | and probe Mix |
| Mix A | 2.5 μL × (n + 1) | 2 μL × (n + 1) |
| Mix B | 2.5 μL × (n + 1) | 2 μL × (n + 1) |
| Mix C | 2.5 μL × (n + 1) | 2 μL × (n + 1) |
| Mix D | 2.5 μL × (n + 1) | 2 μL × (n + 1) |

n = number of reactions (RNA samples plus 2 controls). Prepare enough for 1 extra sample (n + 1) to allow for sufficient overage for the qRT-PCR set-up.

A reaction mix containing all reagents, except for the RNA sample or control templates, should be prepared for the total number of samples and controls to be tested in one run. The Positive Control (PC) and No Template Control (NTC) should be included in each run.

### Suggested Run Layout (96-Well Plate, 384-Well Plate, Tube Strips, or Tubes)

For each reaction, add 4.5 μL of the appropriate assay mix to the plate or tubes. Add up to 5.5 μL of template.

TABLE 6
Suggested plate layout

| | | 1 | 2 | 3 | 4 | 5 | 6 | 7 | 8 | 9 | 10 | 11 | 12 |
|---|---|---|---|---|---|---|---|---|---|---|---|---|---|
| A | Mix A | NTC | S1 | S2 | S3 | S4 | S5 | S6 | S7 | S8 | S9 | S10 | PC |
| B | Mix B | NTC | S1 | S2 | S3 | S4 | S5 | S6 | S7 | S8 | S9 | S10 | PC |
| C | Mix C | NTC | S1 | S2 | S3 | S4 | S5 | S6 | S7 | S8 | S9 | S10 | PC |
| D | Mix D | NTC | S1 | S2 | S3 | S4 | S5 | S6 | S7 | S8 | S9 | S10 | PC |
| E | Mix A | S11 | S12 | S13 | S14 | S15 | S16 | S17 | S18 | S19 | S20 | S21 | S22 |
| F | Mix B | S11 | S12 | S13 | S14 | S15 | S16 | S17 | S18 | S19 | S20 | S21 | S22 |
| G | Mix C | S11 | S12 | S13 | S14 | S15 | S16 | S17 | S18 | S19 | S20 | S21 | S22 |
| H | Mix D | S11 | S12 | S13 | S14 | S15 | S16 | S17 | S18 | S19 | S20 | S21 | S22 |

PC: Positive Control, NTC: No-Template Control (water), S1-22: Samples 1-22.

Table 6 is a suggested plate set-up for a single experiment analyzing 22 unknown samples.

After all reagents have been added to the plate, tightly seal the plate to prevent evaporation. Spin at 1000 rpm for 1 minute to collect all the reagents. Place in the real-time PCR instrument immediately.

### Instrument Set-Up

Set up the PCR reaction thermocycling conditions on validated instruments: Bio-Rad CFX 384 and ABI QuantStudio 5, or ABI 7500 Fast Dx.

### Selection of Detectors

A. For Bio-Rad CFX 384, select all channel
B. For ABI QuantStudio 5 and ABI 7500 Fast Dx, assign individual target in each Mix A, B, C, as “FAM”, and Mix D as “HEX”, respectively.

### 3.4.2. Setup the Cycling Parameters as Shown in Table 7a or Table 7b for Different Instruments

### 3.4.3. Start the Run

For more detailed instructions of setting-up different qPCR instruments, please refer to the Instrument Setting-up and Data Analysis document. This document is available upon request.

TABLE 7a
Bio-Rad CFX 384 cycling parameters

| | Temperature | Time | | Data |
|---|---|---|---|---|
| Step | (° C.) | (Seconds) | Cycles | Collection |
| UNG Incubation | 25 | 120 | 1 | OFF |
| Reverse Transcription | 53 | 600 | 1 | OFF |
| Polymerase Activation | 95 | 120 | 1 | OFF |
| Denaturation | 95 | 3 | X45 | OFF |
| Annealing and | 60 | 30 | | FAM, HEX |
| Extension | | | | |

TABLE 7b
ABI QuantStudio 5 and ABI 7500 Fast Dx** cycling parameters

| | Temperature | Time | Ramp Rate | | Data |
|---|---|---|---|---|---|
| Step | (° C.) | (Seconds) | (° C./s) | Cycles | Collection |
| UNG Incubation | 25 | 120 | 1.6 | 1 | OFF |
| Reverse Transcription | 53 | 600 | 1.6 | 1 | OFF |
| Polymerase Activation | 95 | 120 | 1.6 | 1 | OFF |
| Denaturation | 95 | 3 | 1 | X45 | OFF |
| Annealing and | 60 | 30 | 1 | | FAM, HEX |

**If using ABI FAST 7500 Dx, please use FAST mode with automatic ramp rate settings.

### Data Analysis

Assessment of qPCR Results

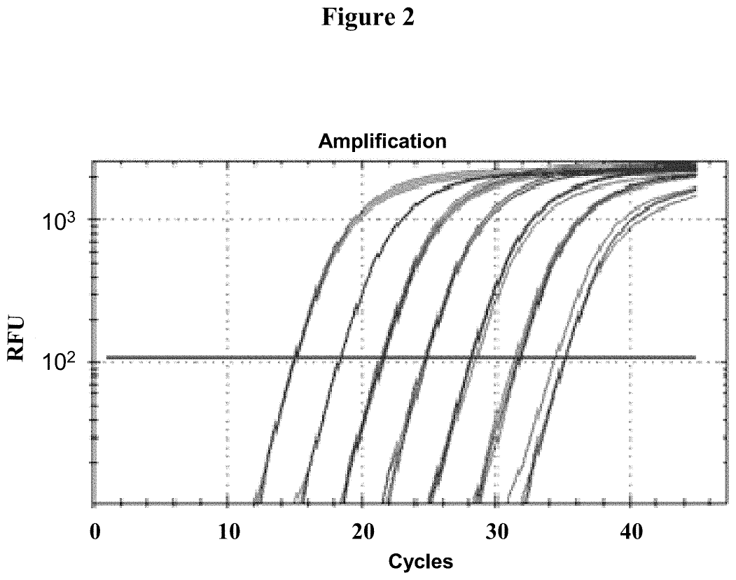

For the Bio-Rad CFX 384, ABI Quant Studio 5 and ABI 7500 Fast Dx, save and analyze the data following the instrument manufacturer's instruction. Adjust the threshold above any background signal to around the middle of the exponential phase of the amplification curve in the log view (e.g. FIG. 2). The procedure chosen for setting the threshold should be used consistently. FIG. 2 is the amplification curve of 10-fold serial dilution of templates showing the threshold setting

### Assessment of the Assay Run

The assay run needs to meet the following criteria to be valid.
Verify that no amplification is observed in the NTC for each of the reaction mixes. Cq should be Undetermined for both FAM and HEX channels
NEC produce a Cq<30 in the Pam channel for Mix D
Positive Controls generates a Cq of 18-26 in the FAM channel for Mix A, Mix B and Mix C

### Interpretation of the Results

Assess the results for each individual assay based on the Cq values in Table 8.
Results are assessed based on Cq values obtained for each individual assay. Tables below show the Cut off values and result interpretation for the assay.

TABLE 8a
Individual assay results - 1

| Target | Cut-Off | Result |
|---|---|---|
| Target Virus Gene | Cq < 38 | POS |
| (A, B, C) | | |
| Target Virus Gene | Cq ≥ 38 | NEG |
| (A, B, C) | | |
| Rnase P (D) | Cq < 36 | Viral RNA input OK |
| Rnase P (D) | Cq ≥ 36 | Viral RNA input fail |

TABLE 8b
Individual assay results - 2

| SARS-CoV2 assay | RNAseP assay | |
|---|---|---|
| (Mix A, B, C) | (NEC) | SARS-CoV2 assay result |
| Ct < 36 | Ct < 30 | Positive |
| 36 ≤ Ct < 37 | Any value | Inconclusive, repeat the test. |
| Ct > 37 | Ct < 30 | Negative |
| Ct undetermined | Ct undetermined | Invalid. Re-isolate RNA then |
| or Ct = 40 | or Ct = 40 | repeats the test. |

### Interpretation of the Results

The Positive control and the NTC (No Template Control) in the kit must function as required to use the Table 9 for interpretation. If the Positive control or the NTC (No Template Control) do not function as required, the test is invalid. All the samples are required to be retested.

TABLE 9a
Interpretation of the results - 1

| orf1ab | N gene | E gene | RNase P | Status | Result | Action |
|---|---|---|---|---|---|---|
| NEG | NEG | NEG | NEG | Invalid | NA | Repeat test one more time. If the |
| | | | | | | repeat result remains invalid, |
| | | | | | | consider collecting new specimen. |
| NEG | NEG | NEG | POS | Valid | SARS-CoV-2 | Report results to healthcare |
| | | | | | Not detected | provider. Consider testing for other |
| | | | | | | respiratory pathogens. |

| | | | | |
|---|---|---|---|---|
| Two or more positive | POS | Valid | SARS-CoV-2 | Report results to healthcare provider |
| | | | Detected | and CDC. |
| Two or more positive | NEG | Valid | SARS-CoV-2 | Report results to healthcare provider |
| | | | Presumptive | and CDC. |
| | | | Detected | |
| One positive | POS or | Valid | SARS-CoV-2 | Repeat test one more time. If the |
| | NEG | | Inconclusive | repeat result remains inconclusive, |
| | | | | contact CDC for guidance. |

TABLE 9b
Interpretation of the results - 2

| SARS-CoV2 assay test result | Intepretion of the results |
|---|---|
| Any two of the three assays | SARS-CoV-2 RNA is detected. |
| (Mix A, B, C) are positive. | |
| Any one of the assays is positive | SARS-CoV-2 RNA is detected. |
| in two different samples collected | |
| from the same subject | |
| All three of the assays | SARS-CoV-2 RNA is not detected. |
| (Mix A, B and C) are negative. | |

### Assay Performance

The performance characteristics of the SARS-CoV-2 assay were established on ABI 7500 Fast Dx and ABI QuantStudio 5 qPCR instruments. Additional tests were performed on BioRadCFX 384.

### Analytical Specificity

The QuantiVirus™ Real-Time PCR Coronavirus (SARS-CoV-2) Detection Test has been designed to detect all publicly available COVID-19 viral RNA sequences. At the same time, the primers and probes were designed in the SARS-CoV-2 virus specific genome region ensuring the specific detection of the SARS-CoV-2 virus. In silico analysis of the SARS-CoV-2 assay design showed that the assay can detect all SARS-CoV2 virus strains and exhibited no cross reactivity with non-SARS-CoV-2 species.

### Precision

Precision studies include intra-run, inter-run, instrument and operator varibility evaluation. The assay precision was assesed by the repeated testing of samples with three different template concentrations.
Inter-assay % CV was established for same lot of reagents tested on the same instrument by the same user.
Intra-assay % CV was established through performance of kit on reference samples run in replicates of nine.
Operator variability was evaluated with one lot of reagents by two operators.

Reproducibility is demonstrated based on % CV of Cq values.

### Intra-Assay Reproducibity

Each assay at three sample template concentrations was repeated 10 times and run on the sampe plate. Average Ct and CV were calculated.

TABLE 10
Intra assay of each target for SARS-Cov-2 detection kit

| SARS-CoV-2 - N Gene (Mix A) | | | | Reference RP (Mix D) | | |
|---|---|---|---|---|---|---|
| Sample | | % | Coefficient | | % | Coefficient |
| concentration | Mean | Replicate | of Variation | Mean | Replicate | of Variation |
| (copies/μL) | Cq | Detection | (%) | Cq | Detection | (%) |
| 50 | 30.987 | 100 | 0.894 | 31.079 | 100 | 1.338 |
| 25 | 31.931 | 100 | 1.019 | 31.775 | 100 | 1.060 |
| 10 | 34.377 | 100 | 2.171 | 33.173 | 100 | 2.886 |
| SARS-CoV-2 - Orf1ab Gene (Mix B) | | | | Reference RP (Mix D) | | |
| Sample | | % | Coefficient | | % | Coefficient |
| concentration | Mean | Replicate | of Variation | Mean | Replicate | of Variation |
| (copies/μL) | Cq | Detection | (%) | Cq | Detection | (%) |
| 50 | 31.336 | 100 | 0.762 | 31.079 | 100 | 1.338 |
| 25 | 32.471 | 100 | 0.802 | 31.775 | 100 | 1.060 |
| 10 | 34.558 | 100 | 2.012 | 33.173 | 100 | 2.886 |
| SARS-CoV-2 - E Gene (Mix C) | | | | Reference RP (Mix D) | | |
| Sample | | % | Coefficient | | % | Coefficient |
| concentration | Mean | Replicate | of Variation | Mean | Replicate | of Variation |
| (copies/μL) | Cq | Detection | (%) | Cq | Detection | (%) |
| 50 | 32.242 | 100 | 2.535 | 31.079 | 100 | 1.338 |
| 25 | 33.101 | 100 | 1.071 | 31.775 | 100 | 1.060 |
| 10 | 35.529 | 100 | 1.527 | 33.173 | 100 | 2.886 |

The Intra assay overall CV was <3% and acceptable for this assay.

### Operator Reproducibility

The assay reactions were set up by two operators using the same lot of reagent and run on the same instrument. Average Ct and CV were calculated.

TABLE 11
Different operator reproducibility

| | Sample | Operator1 | | | Operator2 | | | Overall | | |
|---|---|---|---|---|---|---|---|---|---|---|
| | concentration | Mean | SD | CV | Mean | SD | CV | Mean | SD | CV |
| Assay Target | (copies/μL) | Cq | Cq | (%) | Cq | Cq | (%) | Cq | Cq | (%) |
| SARS-COV-2 - | 5 | 34.1 | 0.9 | 2.6 | 34.4 | 0.7 | 2.2 | 34.2 | 0.2 | 0.6 |
| N Gene | 25 | 32.0 | 0.4 | 1.1 | 31.9 | 0.3 | 1.0 | 32.0 | 0.0 | 0.1 |
| (Mix A) | 50 | 30.9 | 0.2 | 0.7 | 31.0 | 0.3 | 0.9 | 30.9 | 0.1 | 0.3 |
| SARS-COV-2 - | 5 | 34.1 | 0.1 | 0.4 | 34.6 | 0.7 | 0.8 | 34.3 | 0.3 | 0.9 |
| Orf1ab Gene | 25 | 32.5 | 0.2 | 0.5 | 32.5 | 0.3 | 0.8 | 32.5 | 0.0 | 0.0 |
| (Mix B) | 50 | 31.6 | 0.0 | 0.1 | 31.3 | 0.2 | 0.8 | 31.5 | 0.2 | 0.7 |
| SARS-COV-2 - | 5 | 34.9 | 0.8 | 2.4 | 35.5 | 0.5 | 1.5 | 35.2 | 0.4 | 1.2 |
| E Gene | 25 | 33.3 | 0.1 | 0.4 | 33.1 | 0.4 | 1.1 | 33.2 | 0.2 | 0.5 |
| (Mix C) | 50 | 32.2 | 0.3 | 1.0 | 32.2 | 0.8 | 2.5 | 32.2 | 0.0 | 0.1 |
| Reference RP | 5 | 32.7 | 0.6 | 1.7 | 33.2 | 1.0 | 2.9 | 32.9 | 0.3 | 1.0 |
| (Mix D) | 25 | 31.4 | 0.3 | 1.1 | 31.8 | 0.3 | 1.1 | 31.6 | 0.3 | 0.9 |
| | 50 | 30.7 | 0.1 | 0.3 | 31.1 | 0.4 | 1.3 | 30.9 | 0.2 | 0.8 |

Overall CV for two operators is <1.5% and is acceptable for this assay.

### Inter-Instrument Reproducibility

Assay reactions were set up with three replicates and run on three different qPCR instruments including BioRadCFX 384, ABI QS5 and ABI 7500 Fast Dx. Average Ct and CV were calculated.

TABLE 12
Instrument Reproducibility

| | Sample | ABI QS5 | | | Bio-Rad CFX 384 | | | ABI 7500Dx | | | Overall | | |
|---|---|---|---|---|---|---|---|---|---|---|---|---|---|
| | concentration | Mean | SD | CV | Mean | SD | CV | Mean | SD | CV | Mean | SD | CV |
| Assay Target | (copies/μL) | Cq | Cq | (%) | Cq | Cq | (%) | Cq | Cq | (%) | Cq | Cq | (%) |
| SARS-COV-2 - | 5 | 34.1 | 0.9 | 2.6 | 36.6 | 1.0 | 2.7 | 35.7 | 0.9 | 2.6 | 35.5 | 1.3 | 3.6 |
| N Gene | 25 | 32.0 | 0.4 | 1.1 | 34.3 | 0.0 | 0.1 | 33.1 | 0.2 | 0.5 | 33.1 | 1.2 | 3.6 |
| (Mix A) | 50 | 30.9 | 0.2 | 0.7 | 33.2 | 0.2 | 0.6 | 31.5 | 0.1 | 0.3 | 31.8 | 1.2 | 3.7 |
| SARS-COV-2 - | 5 | 34.1 | 0.1 | 0.4 | 34.9 | 0.6 | 1.7 | 34.3 | 0.0 | 0.1 | 34.4 | 0.4 | 1.1 |
| Orf1ab Gene | 25 | 32.5 | 0.2 | 0.5 | 32.9 | 0.2 | 0.7 | 32.3 | 0.1 | 0.3 | 32.6 | 0.3 | 0.9 |
| (Mix B) | 50 | 31.6 | 0.0 | 0.1 | 31.9 | 0.2 | 0.6 | 31.3 | 0.1 | 0.4 | 31.6 | 0.3 | 1.0 |
| SARS-COV-2 - | 5 | 34.9 | 0.8 | 2.4 | 38.1 | 0.8 | 2.1 | 35.7 | 0.7 | 2.0 | 36.2 | 1.7 | 4.6 |
| E Gene | 25 | 33.3 | 0.1 | 0.4 | 35.6 | 0.3 | 0.9 | 33.1 | 0.2 | 0.6 | 34.0 | 1.4 | 4.0 |
| (Mix C) | 50 | 32.2 | 0.3 | 1.0 | 34.5 | 0.2 | 0.6 | 32.1 | 0.1 | 0.2 | 32.9 | 1.3 | 4.1 |
| Reference RP | 5 | 32.7 | 0.6 | 1.7 | 35.2 | 0.7 | 2.0 | 34.9 | 1.0 | 2.8 | 34.3 | 1.4 | 4.0 |
| (Mix D) | 25 | 31.4 | 0.3 | 1.1 | 33.7 | 0.3 | 0.8 | 33.0 | 0.3 | 1.0 | 32.7 | 1.2 | 3.6 |
| | 50 | 30.7 | 0.1 | 0.3 | 33.3 | 0.2 | 0.5 | 32.1 | 0.2 | 0.5 | 32.1 | 1.3 | 4.0 |

The results indicate that three instruments have <5% CV and is acceptable.

### Analytic Sensitivity and Limit of Detection (LOD)

To determine the Limit of Detection (LoD) and analytical sensitivity of the kit, studies were performed using serial dilutions of analyte and the LoD was determined to be the lowest concentration of template that could reliably be detected with 95% of all tested positive. LoD of each target assay in the QuantiVirus™ SARS-CoV-2 Test were conducted and verified using SeraCare AccuPlex SARS-CoV-2 Reference Material Kit (Cat #0505-0126). Non-infectious viral particles from the AccuPlex SARS-CoV-2 Reference Material Kit were spiked in sputum at various concentrations (50 copies/mL, 100 copies/mL, 150 copies/mL, 200 copies/mL and 300 copies/mL) diluted from the stock concentration of 5000 copies/mL. Real-time RT-PCR assay was performed with the provided kit reagents and tested on ABI QS5 and ABI 7500 Fast Dx PCR instruments.

The process of preparing the spiked samples was as follows:
1. Sputum samples were collected following the CDC Guidance for collecting sputum samples.
2. 100 μL of the sputum was mixed with 100 uL lysis buffer at 1:1 ratio.
3. SARS-CoV-2 viral particles carrying N gene, ORF 1 ab and E gene (SeraCare AccuPlex Reference material Kit, cat #0505-0126) were added to 200 uL sputum mix separately at the following concentrations: 50 copies/mL, 100 copies/mL, 150 copies/mL, 200 copies/mL, 300 copies/mL.
4. Process the 200 μL spiked samples from step #3 above using the Thermo Fisher viral RNA extraction kit (PureLink™ Viral RNA/DNA Mini Kit, cat #12280050). Elute the extracted RNA to 25 μL with sterile RNase-free water.
5. Take 5.5 μL purified RNA samples for each reaction and run the qRT-PCR using the QuantiVirus™ SARS-CoV-2 Test kits.

We tested 50 copies/mL, 100 copies/mL, 150 copies/mL, 200 copies/mL and 300 copies/mL of viral RNA in sputum sample. Table 13 shows N gene, ORF lab and E gene can be detected down to 100 copies/mL. The negative control did not show any signal (Ct >39). Positive control showed Ct. <24. Positive sample cutoff Ct value was determined to be Ct<38. At 100 copies/mL testing level, its average Ct in N gene was 34.2, in ORFlab was 35.7 and in E gene was 37.7. These data indicate that the assay sensitivity (LOD) is 100 copies/mL.

TABLE 13
Assay Sensitivity by Spiking viral RNA in Sputum

| Copies/mL | Rxn A- NGene | | | RxnB - Orf1ab Gene | | | RxnC - E Gene | | | RxnD - RP Gene | | |
|---|---|---|---|---|---|---|---|---|---|---|---|---|
| 300 | 33.2 | 33.0 | 32.6 | 33.8 | 33.6 | 35.1 | 35.3 | 35.3 | 35.1 | 45.0 | 45.0 | 38.1 |
| 200 | 33.2 | 34.0 | 34.1 | 34.5 | 35.0 | 35.0 | 36.0 | 35.5 | 35.4 | 45.0 | 45.0 | 45.0 |
| 150 | 34.1 | 33.7 | 33.2 | 34.2 | 34.2 | 34.4 | 36.6 | 35.5 | 35.7 | 45.0 | 45.0 | 45.0 |
| 100 | 35.3 | 34.2 | 33.2 | 36.0 | 35.2 | 35.8 | 37.5 | 36.5 | 39.2 | 45.0 | 45.0 | 45.0 |
| 50 | 36.9 | 37.1 | 35.0 | 35.5 | 36.8 | 36.5 | 37.1 | 37.5 | 36.9 | 45.0 | 45.0 | 45.0 |
| PC | 23.8 | 23.6 | 23.7 | 23.4 | 23.4 | 23.5 | 23.6 | 23.5 | 23.4 | 45.0 | 45.0 | 45.0 |
| NTC | 45.0 | 45.0 | 45.0 | 39 | 45.0 | 45.0 | 45.0 | 45.0 | 45.0 | 45.0 | 45.0 | 45.0 |

| Statistics | Avg | Std | 99% CI | Avg | Std | 99% CI | Avg | Std | 99% CI | Avg | Std | 99% CI |
|---|---|---|---|---|---|---|---|---|---|---|---|---|
| 300 | 32.9 | 0.3 | 0.7 | 34.2 | 0.7 | 1.8 | 35.2 | 0.1 | 0.3 | 42.7 | 3.2 | 8.3 |
| 200 | 33.8 | 0.4 | 0.9 | 34.8 | 0.2 | 0.6 | 35.6 | 0.3 | 0.7 | 45.0 | 0.0 | 0.0 |
| 150 | 33.7 | 0.4 | 1.0 | 34.3 | 0.1 | 0.3 | 35.9 | 0.5 | 1.2 | 45.0 | 0.0 | 0.0 |
| 100 | 34.2 | 0.8 | 2.2 | 35.7 | 0.3 | 0.8 | 37.7 | 1.1 | 2.8 | 45.0 | 0.0 | 0.0 |
| 50 | 36.3 | 0.9 | 2.4 | 36.3 | 0.6 | 1.4 | 37.3 | 0.4 | 1.1 | 45.0 | 0.0 | 0.0 |
| PC | 23.7 | 0.1 | 0.2 | 23.4 | 0.1 | 0.1 | 23.5 | 0.1 | 0.3 | 45.0 | 0.0 | 0.0 |
| NTC | 45.0 | 0.0 | 0.0 | 43.0 | 2.9 | 7.5 | 45.0 | 0.0 | 0.0 | 45.0 | 0.0 | 0.0 |

The LOD was confirmed by testing 1×LoD of viral RNA with 20 replicates. The LoD was determined to be the lowest concentration (copies/mL) at which >95% (19/20) of the 20 replicates were tested as positive. Again, viral RNA was spiked in sputum, extracted and tested by the QuantiVirus SARS-Cov-2 RT-qPCR. Average Ct from 20 samples for N gene, ORF lab and E gene were between Ct 33-36 with 95% CI. Twenty samples with 100 copies/mL viral RNA was detectable in this experiment. Its correct call rate was 95-100% (Table 14).

TABLE 14
Twenty Replicate Test for LOD Confirmation

| Viral RNA | | | | |
|---|---|---|---|---|
| Concentration (Copy/mL) | 100 Copies/mL | | | |
| Target Gene | N Gene | Orf Gene | E Gene | RP Gene |
| 1 | 34.0 | 35.4 | 36.3 | 23.6 |
| 2 | 33.7 | 33.9 | 36.5 | 23.3 |
| 3 | 34.4 | 34.1 | 34.9 | 26.7 |
| 4 | 33.8 | 35.5 | 35.2 | 28.5 |
| 5 | 34.3 | 36.0 | 37.2 | 24.9 |
| 6 | 33.1 | 35.0 | 34.8 | 24.1 |
| 7 | 33.7 | 35.3 | 35.0 | 26.7 |
| 8 | 34.4 | 34.9 | 36.1 | 24.3 |
| 9 | 33.2 | 35.7 | 34.7 | 28.0 |
| 10 | 32.6 | 34.1 | 36.4 | 24.3 |
| 11 | 33.1 | 34.2 | 34.7 | 23.3 |
| 12 | 34.1 | 35.0 | 35.4 | 24.3 |
| 13 | 33.0 | 35.4 | 35.2 | 24.6 |
| 14 | 36.1 | 38.0 | 38.0 | 27.1 |
| 15 | 33.5 | 35.4 | 34.0 | 24.9 |
| 16 | 33.2 | 35.1 | 34.7 | 23.3 |
| 17 | 34.8 | 34.5 | 35.1 | 24.5 |
| 18 | 32.7 | 35.3 | 35.4 | 25.3 |
| 19 | 33.5 | 35.2 | 36.2 | 26.4 |
| 20 | 33.5 | 34.7 | 35.9 | 23.8 |
| Avg | 33.7 | 35.1 | 35.6 | 25.1 |
| Std | 0.8 | 0.9 | 0.9 | 1.6 |
| 99% CI | 2.0 | 2.2 | 2.4 | 4.0 |
| NTC | 45.0 | 45.0 | 45.0 | 45.0 |
| PC | 22.8 | 22.4 | 22.3 | 45.0 |

*NTC—no target control; PC—positive control

The LOD was confirmed by testing 1×LoD of viral RNA with 20 replicates. The LoD was determined to be the lowest concentration (copies/ml) at which >95% (19/20) of the 20 replicates were tested as positive. The data confirmed the assay analytical sensitivity was 200 copies/mL for this assay in ABI QS5 (Table 15a) and 100 copies/mL for this assay in ABI7500Dx (Table 15b)

### LoD for ABI QuantStudio 5

The data confirmed the assay analytical sensitivity was 200 copies/mL for ABI QuantStudio 5.

TABLE 15a
Summary of Twenty Replicates for Assay Sensitivity (ABI QuantStudio 5)

| Target | RNA (copy/mL) | Total | AVE Ct | SD | CV | Positive | Negative | Call Rate |
|---|---|---|---|---|---|---|---|---|
| N GENE | 100 copies/mL | 20 | 33.73 | 0.79 | 0.02 | 20 | 0 | 100% |
| ORF1ab | 100 copies/mL | 20 | 35.13 | 0.87 | 0.02 | 20 | 0 | 100% |
| E GENE | 150 copies/mL | 20 | 37.31 | 1.9 | 0.05 | 18 | 2 | 90% |
| | 200 copies/mL | 20 | 36.77 | 2.0 | 0.05 | 19 | 1 | 95% |

### LoD for ABI 7500 Fast Dx

The data confirmed the assay analytical sensitivity was 100 copies/mL for ABI 7500 Fast Dx.

TABLE 15b
Summary of Twenty Replicates for Assay Sensitivity (ABI 7500 Fast Dx)

| Target | RNA (copy/mL) | Total | AVE Ct | SD | CV | Positive | Negative | Call Rate |
|---|---|---|---|---|---|---|---|---|
| N GENE | 100 copies/mL | 20 | 33.73 | 0.79 | 0.02 | 20 | 0 | 100% |
| ORF1ab | 100 copies/mL | 20 | 35.13 | 0.87 | 0.02 | 20 | 0 | 100% |
| E GENE | 100 copies/mL | 20 | 35.59 | 0.95 | 0.03 | 20 | 0 | 100% |

### Cross-Reactivity

The QuantiVirus™ SARS-CoV-2 Test kit has been designed to detect all publicly available SARS-CoV-2 strains. At the same time, the primers and probes were designed in the SARS-CoV-2 virus specific genome region ensuring the specific detection of the SARS-CoV-2 viral RNA. In silico analysis of the SARS-CoV2 assay design were performed and compared to common respiratory flora and other viral pathogens from the same genetic family as SARS-CoV-2 according to the Recommended List of Organisms to be analyzed in silico (see Table 16 and 17) or by direct wet lab testing (Table 18).

TABLE 16
List of organisms tested for cross-reactivity by in silico analysis

| # | Organism |
|---|---|
| 1 | Human coronavirus 229E |
| 2 | Human coronavirus OC43 |
| 3 | Human coronavirus HKU1 |
| 4 | Human coronavirus NL63 |
| 5 | SARS-coronavirus |
| 6 | MERS-coronavirus |
| 7 | Adenovirus |
| 8 | Human Metapneumovirus (hMPV) |
| 9 | Parainfluenza virus 1-4 |
| 10 | Influenza A |
| 11 | Influenza B |
| 12 | Enterovirus |
| 13 | Respiratory Syncytial Virus A |
| 14 | Rhinovirus |
| 15 | Enterovirus |
| 16 | Chlamydia pneumoniae |
| 17 | Haemophilus influenzae |
| 18 | Legionella pneumophila |
| 19 | Mycobacterium tuberculosis |
| 20 | Streptococcus pneumoniae |
| 21 | Streptococcus pyogenes |
| 22 | Bordetella pertussis |
| 23 | Candida albicans |
| 24 | Pseudomonas aeruginosa |
| 25 | Staphylococcus epidermis |
| 26 | Staphylococcus salivarius |

All of other homologies were not significant for the pair of primers and probes in order to predict a in silico false positive result. Results of the In Silico Sequence homology analysis for the common respiratory organisms demonstrated, that one organism—SARS-coronavirus (MK062184.1) showed significant homology (>80%) for the primers and probes of 2 out of the 3 genes in our assay. The analysis in a tabular format is shown in Table 173. All results that yield significant homology (>80%) are highlighted in the Table 17. All other homologies were not significant for the pair of primers in order to predict a in silico false positive result.

TABLE 17
In Silico Sequence Homology against specific Corona Viruses and
Common Respiratory Pathogens (Search conducted on 25th Mar. 2020)

| | | | | | % Homology | | |
|---|---|---|---|---|---|---|---|
| | | | | | E gene | | |
| | | | | | | Forward | Reverse |
| | | | | | Probe- | Primer- | Primer- |
| # | Organism | | | Query | WHnCoVPr4 | WHnCoVF4 | WHnCoVR4 |
| 1 | Human coronavirus 229E | | | AF304460.1 | *NSH | *NSH | *NSH |
| 2 | Human coronavirus OC43 | | | MN310478.1 | *NSH | *NSH | *NSH |
| 3 | Human coronavirus HKU1 | | | KY674943.1 | *NSH | *NSH | *NSH |
| 4 | Human coronavirus NL63 | | | MN306040.1 | 40.7 | 57.9 | *NSH |
| 5 | SARS-coronavirus | | | MK062184.1 | 81.5 | 100.0 | 90.9 |
| 6 | MERS-coronavirus | | | KJ556336.1 | *NSH | *NSH | 50.0 |
| 7 | Adenovirus | | | MK241690.1 | 40.7 | *NSH | *NSH |
| 8 | Human Metapneumovirus (hMPV) | | | NC_039199.1 | *NSH | *NSH | *NSH |
| 9 | Parainfluenza virus 1 | | | KX639498.1 | *NSH | *NSH | *NSH |
| 10 | Parainfluenza virus 2 | | | KM190939.1 | *NSH | *NSH | *NSH |
| 11 | Parainfluenza virus 3 | | | NC_001796.2 | *NSH | *NSH | 50.0 |
| 12 | Parainfluenza virus 4 | | | NC_021928.1 | *NSH | *NSH | *NSH |
| 13 | Enterovirus | | | EU870491.1 | *NSH | *NSH | *NSH |
| 14 | Respiratory Syncytial Virus A | | | LC488177.1 | 48.1 | *NSH | *NSH |
| 15 | Rhinovirus | | | FJ445119.1 | *NSH | *NSH | *NSH |
| 16 | Chlamydia pneumoniae | | | CP001713.1 | 51.9 | *NSH | 68.2 |
| 17 | Haemophilus influenzae | | | LS483480.1 | 48.1 | 63.2 | 54.5 |
| 18 | Legionella pneumophila | | | LT632617.1 | 44.4 | 68.4 | 63.6 |
| 19 | Mycobacterium tuberculosis | | | CP049108.1 | 51.9 | 84.2 | 54.5 |
| 20 | Streptococcus pneumoniae | | | AP019192.2 | 44.4 | 68.4 | 72.7 |
| 21 | Streptococcus pyogenes | | | CP035430.1 | 48.1 | *NSH | *NSH |
| 22 | Bordetella pertussis | | | CP011448.1 | 44.4 | *NSH | 59.1 |
| 23 | Candida albicans | | | CP032018.1 | 77.8 | 73.7 | 59.1 |
| 24 | Pseudomonas aeruginosa | | | CP013113.1 | *NSH | *NSH | 68.2 |
| 25 | Staphylococcus epidermis | | | KY750253.1 | *NSH | *NSH | *NSH |
| 26 | Staphylococcus salivarius | | | BX571856.1 | 59.3 | *NSH | 68.2 |
| 27 | Mycoplasma pneumoniae | | | NC_000912.1 | 44.4 | 57.9 | 54.5 |
| 28 | Pneumocystis jirovecii | | | ASM33397v2 | 44.4 | 63.2 | 68.2 |
| 29 | Influenza A | | | EF190975.1 | *NSH | *NSH | 50.0 |
| 30 | Influenza A | | | EF190971.1 | *NSH | *NSH | *NSH |
| 31 | Influenza A | | | EF190978.1 | *NSH | *NSH | *NSH |
| 32 | Influenza A | | | EF190977.1 | *NSH | *NSH | *NSH |
| 33 | Influenza A | | | EF190976.1 | *NSH | *NSH | *NSH |
| 34 | Influenza A | | | EF190974.1 | *NSH | *NSH | *NSH |
| 35 | Influenza A | | | EF190973.1 | *NSH | *NSH | *NSH |
| 36 | Influenza A | | | EF190972.1 | *NSH | *NSH | *NSH |
| 37 | Influenza B | | | NC_002211.1 | *NSH | *NSH | *NSH |
| 38 | Influenza B | | | NC_002210.1 | *NSH | *NSH | *NSH |
| 39 | Influenza B | | | NC_002209.1 | *NSH | *NSH | *NSH |
| 40 | Influenza B | | | NC_002208.1 | *NSH | *NSH | 68.2 |
| 41 | Influenza B | | | NC_002207.1 | *NSH | *NSH | *NSH |
| 42 | Influenza B | | | NC_002206.1 | *NSH | *NSH | *NSH |
| 43 | Influenza B | | | NC_002205.1 | *NSH | *NSH | *NSH |
| 44 | Influenza B | | | NC_002204.1 | *NSH | *NSH | *NSH |
| | | % Homology | | | | | |
| | | N gene | | | orf1ab | | |
| | | | Forward | Reverse | | Forward | Reverse |
| | | Probe- | Primer- | Primer- | Probe- | Primer- | Primer- |
| | # | WHnCoVPr2 | WHnCoVF2 | WHnCoVR2a | WHnCoVPr3 | WHnCoVF3 | WHnCoVR3 |
| | 1 | *NSH | 50.0 | *NSH | 37.9 | *NSH | 90.5 |
| | 2 | *NSH | 50.0 | *NSH | 37.9 | *NSH | *NSH |
| | 3 | 59.1 | 54.5 | *NSH | 37.9 | 85.0 | 85.7 |
| | 4 | *NSH | 54.5 | *NSH | 37.9 | 90.0 | 66.7 |
| | 5 | 100.0 | 86.4 | 84.2 | 72.4 | 60.0 | *NSH |
| | 6 | *NSH | *NSH | *NSH | 44.8 | 80.0 | 90.5 |
| | 7 | *NSH | *NSH | *NSH | *NSH | *NSH | *NSH |
| | 8 | *NSH | *NSH | *NSH | *NSH | *NSH | *NSH |
| | 9 | *NSH | *NSH | *NSH | *NSH | *NSH | *NSH |
| | 10 | *NSH | *NSH | *NSH | *NSH | *NSH | 52.4 |
| | 11 | *NSH | *NSH | *NSH | *NSH | *NSH | 57.1 |
| | 12 | *NSH | *NSH | *NSH | *NSH | *NSH | *NSH |
| | 13 | *NSH | *NSH | *NSH | *NSH | *NSH | *NSH |
| | 14 | *NSH | *NSH | *NSH | *NSH | *NSH | *NSH |
| | 15 | *NSH | *NSH | *NSH | *NSH | *NSH | *NSH |
| | 16 | 63.6 | 50.0 | 63.2 | 44.8 | 85.0 | 52.4 |
| | 17 | 63.6 | 72.7 | 57.9 | 65.5 | 75.0 | 57.1 |
| | 18 | 54.5 | 95.5 | 63.2 | 41.4 | 70.0 | 52.4 |
| | 19 | 59.1 | *NSH | 73.7 | 51.7 | *NSH | 57.1 |
| | 20 | 68.2 | 68.2 | 78.9 | 48.3 | 60.0 | 57.1 |
| | 21 | 63.6 | 72.7 | 63.2 | 41.4 | 85.0 | 76.2 |
| | 22 | 50.0 | *NSH | *NSH | 37.9 | *NSH | *NSH |
| | 23 | 59.1 | 77.3 | 57.9 | 41.4 | 60.0 | 76.2 |
| | 24 | 54.5 | 59.1 | 89.5 | *NSH | *NSH | *NSH |
| | 25 | *NSH | *NSH | *NSH | *NSH | *NSH | *NSH |
| | 26 | 59.1 | 72.7 | 63.2 | 48.3 | 85.0 | 61.9 |
| | 27 | 54.5 | 63.6 | 57.9 | 48.3 | 60.0 | 57.1 |
| | 28 | 63.6 | 50.0 | 63.2 | 41.4 | 80.0 | *NSH |
| | 29 | *NSH | *NSH | *NSH | *NSH | *NSH | *NSH |
| | 30 | *NSH | *NSH | *NSH | *NSH | *NSH | *NSH |
| | 31 | *NSH | *NSH | *NSH | *NSH | *NSH | *NSH |
| | 32 | *NSH | *NSH | *NSH | *NSH | *NSH | *NSH |
| | 33 | *NSH | *NSH | *NSH | *NSH | *NSH | *NSH |
| | 34 | *NSH | *NSH | *NSH | *NSH | *NSH | *NSH |
| | 35 | *NSH | *NSH | *NSH | *NSH | *NSH | *NSH |
| | 36 | *NSH | *NSH | *NSH | *NSH | *NSH | *NSH |
| | 37 | 54.5 | *NSH | *NSH | *NSH | *NSH | *NSH |
| | 38 | *NSH | *NSH | *NSH | *NSH | *NSH | *NSH |
| | 39 | *NSH | *NSH | *NSH | *NSH | *NSH | *NSH |
| | 40 | *NSH | *NSH | *NSH | *NSH | *NSH | *NSH |
| | 41 | *NSH | *NSH | *NSH | *NSH | *NSH | *NSH |
| | 42 | *NSH | *NSH | 57.9 | *NSH | *NSH | *NSH |
| | 43 | *NSH | *NSH | *NSH | *NSH | *NSH | *NSH |
| | 44 | *NSH | *NSH | *NSH | *NSH | *NSH | *NSH |

*NSH—No Significant Homology; Yellow Highlights - Homology more than 80%

Results of in Silico analysis demonstrates that there is significant homology between the SARS-coronavirus (MK062184.1) and our assay primer/probes for N gene and E gene. Therefore, the cross reactivity with SARS-coronavirus (MK062184.1) was tested by wet laboratory experiments.

We have tested the cross-reactivity in wet lab. MERS-coronavirus, SARS-CoV coronavirus samples were ordered from IDT and NATtrol Respiratory Validation Panel from ZeptoMetrix (cat #NATRVP-3). RNA/DNA were extracted from high titer stocks of the potentially cross-reacting microorganisms (estimated 1.0E+05 unites/mL),RNA/DNA were extracted from 100 μL microorganisms stocks using the Thermo Fisher viral RNA extraction kit (PureLink™ Viral RNA/DNA Mini Kit, cat #12280050) and Qiagen QIAamp DNA Mini Kit (Cat #. 51304). Elute the extracted sample RNA/DNA to 100 μL with sterile RNase-free water. Take 5.5 μL purified RNA/DNA samples for each reaction and run the qRT-PCR with QuantiVirus SARS-CoV-2 Test Kit. The cross-reactivity testing results are summarized in Table x. The tests were run in triplicates. All the test controls passed (Positive control for three targets passed (Ct<25), No target control passed (Ct >45), Extraction control has RP ˜Ct 28). The tested organisms all show negative for the three targeted genes of SARS-CoV-2, suggesting there is no cross-reactivity between SARS-CoV-2 and the organisms tested.

TABLE 18
Summary of Cross- Reactivity Between SARS-CoV-2 Kit and Organisms tested

| | | N gene | | Orf gene | | | E gene | | | RP IC | | |
|---|---|---|---|---|---|---|---|---|---|---|---|---|
| Organisms | Avg | Std | 99% CI | Avg | Std | 99% CI | Avg | Std | 99% CI | Avg | Std | 99% CI |
| Coronavirus 229E | 45 | 0 | 0 | 45 | 0 | 0 | 45 | 0 | 0 | 45 | 0 | 0 |
| Coronavirus HKU-1 | 45 | 0 | 0 | 45 | 0 | 0 | 45 | 0 | 0 | 42 | 4 | 11 |
| Coronavirus NL63 | 45 | 0 | 0 | 45 | 0 | 0 | 45 | 0 | 0 | 45 | 0 | 0 |
| Coronavirus OC43 | 45 | 0 | 0 | 45 | 0 | 0 | 45 | 0 | 0 | 45 | 0 | 0 |
| Influenza A H1N1pdm | 45 | 0 | 0 | 45 | 0 | 0 | 45 | 0 | 0 | 45 | 0 | 0 |
| Influenza AH1 | 45 | 0 | 0 | 45 | 0 | 0 | 45 | 0 | 0 | 42 | 4 | 10 |
| Influenza AH3 | 45 | 0 | 0 | 45 | 0 | 0 | 45 | 0 | 0 | 45 | 0 | 0 |
| Influenza B | 45 | 0 | 0 | 45 | 0 | 0 | 45 | 0 | 0 | 45 | 0 | 0 |
| Parinfluenza 1 | 45 | 0 | 0 | 45 | 0 | 0 | 45 | 0 | 0 | 45 | 0 | 0 |
| Parinfluenza 2 | 45 | 0 | 0 | 45 | 0 | 0 | 45 | 0 | 0 | 40 | 4 | 9 |
| Parinfluenza 3 | 45 | 0 | 0 | 45 | 0 | 0 | 45 | 0 | 0 | 42 | 5 | 12 |
| Parinfluenza 4 | 45 | 0 | 0 | 45 | 0 | 0 | 45 | 0 | 0 | 45 | 0 | 0 |
| Adenovirus3 | 45 | 0 | 0 | 45 | 0 | 0 | 45 | 0 | 0 | 45 | 0 | 0 |
| Metapneumovirus | 45 | 0 | 0 | 45 | 0 | 0 | 45 | 0 | 0 | 45 | 0 | 0 |
| Rhinovirus | 45 | 0 | 0 | 45 | 0 | 0 | 45 | 0 | 0 | 42 | 5 | 12 |
| RSV A | 45 | 0 | 0 | 45 | 0 | 0 | 45 | 0 | 0 | 45 | 0 | 0 |
| B. pertussis | 40 | 3 | 9 | 45 | 0 | 0 | 45 | 0 | 0 | 45 | 0 | 0 |
| C. pneumoniae | 40 | 2 | 6 | 42 | 4 | 10 | 45 | 0 | 0 | 45 | 0 | 0 |
| M. pneumoniae | 42 | 4 | 10 | 45 | 0 | 0 | 45 | 0 | 0 | 45 | 0 | 0 |
| MERS-coronavirus | 45 | 0 | 0 | 45 | 0 | 0 | 45 | 0 | 0 | 45 | 0 | 0 |
| SARS-coronavirus | 45 | 0 | 0 | 45 | 0 | 0 | 45 | 0 | 0 | 45 | 0 | 0 |
| PC | 25 | 0 | 0 | 24 | 0 | 0 | 25 | 0 | 0 | 45 | 0 | 0 |
| NTC | 45 | 0 | 0 | 45 | 0 | 0 | 45 | 0 | 0 | 45 | 0 | 0 |
| EC | | | | | | | | | | 28 | | |

*PC—positive control; NTC—no target control; EC—extraction control

Clinical Evaluation (In Vitro Transcribed viral RNA spiked into sputum)

### Clinical Evaluation on ABI QuantStudio 5

Clinical evaluation of the QuantiVirus™ SARS-CoV-2 Test kit was conducted with contrived sputum specimens including 60 positive and 38 negative samples (Table 13a). Sputum samples were mixed with the lysis buffer from the extraction kit at 1:1 ratio before spiking in non-infectious viral particles (SeraCare AccuPlex SARS-CoV-2 Reference Material Kit, Cat #0505-0126).

Sputum samples (20 samples) were contrived with non-infectious viral particles templates at 0.75×LoD (150 copies/mL), 20 samples at 1×LoD (1×200 copies/mL) and 10 sputum samples were spiked with non-infectious virus at 1.5×LoD (300 copies/mL) and another 10 sputum samples were spiked at the concentration of 2.5×LoD (500 copies/mL). Viral RNA was extracted from spiked samples and tested blindly with the QuantiVirus™ SARS-CoV-2 RT-qPCR.

Data show that there is 95% agreement with the spiked sample at 1×LoD (1×200 copies/mL), and 100% agreement at all other concentrations including 300 copies/mL and 500 copies/mL (Table 19a). For negative control, there was one sample excluded due to contamination. The remaining 37 samples were negative.

TABLE 19a
Contrived clinical sample evaluation with in vitro transcribed RNA (QuantStudio 5)

| Specimen | | SARS-CoV-2 | | | Performance | |
|---|---|---|---|---|---|---|
| Type | Viral RNA Spiked | Positive | Negative | Total | Agreement | 95% CI |
| viral RNA + | 150 copies/mL | 18 | 2 | 20 | 90% | 69.9-97.2% |
| sputum | (0.75x LoD) | | | | | |
| | 200 copies/mL | 19 | 1 | 20 | 95% | 76.4-99.1% |
| | (1x LoD) | | | | | |
| | 300 copies/mL | 10 | 0 | 10 | 100% | 72.3-100% |
| | (1.5x LoD) | | | | | |
| | 500 copies/mL | 10 | 0 | 10 | 100% | 72.3-100% |
| | (2.5x LoD) | | | | | |
| H2O + | 0 copy/mL | 0 | 37 | 37 | 100% | 90.6-100% |
| sputum | | | | | | |

### Clinical Evaluation on ABI 7500 Fast Dx

Clinical evaluation of the QuantiVirus™ SARS-CoV-2 Test kit was conducted with contrived sputum specimens including 40 positive and 38 negative samples (Table 13b). Sputum samples were mixed with the lysis buffer from the extraction kit at 1:1 ratio before spiking in non-infectious viral particles (SeraCare AccuPlex SARS-CoV-2 Reference Material Kit, Cat #0505-0126).

Sputum samples (20 samples) were contrived with non-infectious viral particles templates at 1×LoD (1×100 copies/mL) and 10 sputum samples were spiked with non-infectious virus at 3×LoD (3×100 copies/mL) and another 10 sputum samples were spiked at the concentration of 5×LoD (5×100 copies/mL). Viral RNA was extracted from spiked samples and tested blindly. Data show that there is 100% agreement with the spiked sample at 1×LoD (1×100 copies/mL), and 100% agreement at all other concentrations including 3×LoD and 5×LoD (Table 19b). For negative control, there was one sample excluded due to contamination. The remaining 37 samples were negative

TABLE 19b
Contrived clinical sample evaluation with
in vitro transcribed RNA (ABI 7500 Fast Dx)

| Specimen | | SARS-CoV-2 | | | Performance | |
|---|---|---|---|---|---|---|
| Type | Viral RNA Spiked | Positive | Negative | Total | Agreement | 95% CI |
| viral RNA + | 100 copies/mL | 20 | 0 | 20 | 100% | 83.9-100% |
| sputum | (1x LoD) | | | | | |
| | 300 copies/mL | 10 | 0 | 10 | 100% | 72.3-100% |
| | (3x LoD) | | | | | |
| | 500 copies/mL | 10 | 0 | 10 | 100% | 72.3-100% |
| | (5xLoD) | | | | | |
| H2O + | 0 copy/mL | 0 | 37 | 37 | 100% | 90.6-100% |
| sputum | | | | | | |

### Actual Patient Samples

DiaCarta has tested 5 real patient samples with our QuantiVirus SARS-CoV-2 test using the ABI 7,500 Dx Fast instrument at DiaCarta's Laboratory. We compared our results with the results of the Abbott RealTime SARS-CoV-2 kit used on the M2000 instrument located at the San Francisco Veterans Administration Hospital (Table 20). Our results were also compared to the CDC 2019-nCoV Real-Time RT-PCR kit used on the ABI 7,500 Dx instrument located at the University of California at San Francisco. Our kit detected COVID-19 in two patient samples and did not detect three patient samples. The results from our test kit are the same as those from the Abbott and CDC test kits. The concordance is 100% with the two test kits and two instruments.

TABLE 20
Testing of 5 Real Patient Samples with kit of the invention on ABI 7500
Dx Fast instrument and comparing the test data with the data acquired by
using Abbott RealTime SARS-CoV-2 kit on M2000 instrument (SFVA Hospital)
and CDC 2019-nCoV Real-Time RT-PCR kit on ABI 7500 Dx instrument (UCSF)

| | | Abbott | CDC 2019-nCoV | |
|---|---|---|---|---|
| | | RealTime | Real-Time | DiaCarta |
| Sample | Additional | SARS-CoV-2 | RT-PCR | SARS-CoV-2 |
| ID | Information | SFVA Data | UCSF Data | Test |
| Sample 1 | Patient sample | Not Detected | Not Detected | Not detected |
| Sample 2 | Patient sample | Not Detected | Not Detected | Not detected |
| Sample 3 | Patient sample | Not Detected | Not Detected | Not detected |
| Sample 4 | Patient sample (1:10 diluted) | Detected | Detected | Detected |
| Sample 5 | Patient sample (1:100,000 diluted) | Detected | Detected | Detected |

### Shelf-Life

Based on individual component shelf life and other in-house stability data for similar products, the approximate shelf life of the kit is estimated to be 12 months.

The product contains no substances which at their given concentration, are considered to be hazardous to health.

### Example II

### Methods

### Saliva Clinical Specimens

Clinical samples were collected from patients who had previously been tested positive for SARS-CoV-2. The QuantiVirus™ Saliva Collection Kit (DiaCarta, Inc. cat #DC-11-0021) (see FIG. 1) was used for saliva collection, following the kit insert instructions and under the supervision of healthcare providers. There was no eat or drink 30 minutes before saliva sample collection. Each saliva sample contained 2 mL liquid saliva and 2 mL viral transport media. Saliva samples were refrigerated and processed for testing within 24 hours after collection. Detailed procedures used are listed below:

1. Do not eat or drink 30 minutes before collecting saliva samples.

2. Mark the 2-mL line with a marker. (The collection line is difficult to see if unmarked.)

3. Take the collection tube with the mouth adapter piece and press the tip of your tongue against the roof of your mouth or tooth root to enrich saliva. Spit saliva until it fills to the 2-mL mark.

4. Unscrew the blue topped preservative liquid and pour it into the collection tube with mouth adapter piece, while keeping tube in upright position.

5. Screw off mouth adapter piece. Cover the collection tube with the pink top piece, and mix up and down for at least 5 times.

6. Make sure your tube is labelled, minimally with your name and date of birth, if handing it to a health care professional.

7. Providers and medical personnel can now label the sample following the labelling instructions below. Wipe saliva kit Clean and place into a clean, small biohazard transport bag.

### Viral RNA Extraction

MGI's automatic RNA/DNA extraction instrument MGISP-960 (MGI Tech Co., China) was used for the SARS-CoV-2 viral RNA extraction according to the manufacturer's instructions, for which 200 μL of saliva sample was used. For each batch of clinical samples to be tested, an extraction control (EC) was included (spike 20 μL of EC from the QuantiVirus™ SARS-CoV-2 multiplex kit into 180 μL sterile RNase-free water). The clinical samples and spiked EC were processed and extracted on the MGI platform. The extraction output is RNA in 30-50 μL RNase-free water, 5.5 of which is used for the PCR reaction per test. The turnaround time from sample extraction to PCR final report is around 4 hrs. Precautions were taken while handling extracted RNA samples to avoid RNA degradation. Extracted RNA samples were stored at −80° C. if not immediately used for RT-PCR.

Real-Time Reverse-Transcription PCR (rRT-PCR)
The total volume of one RT-PCR reaction for all targets is 10 μL, including 5.5 μL of RNA, 2.0 μL of 5× primer and probe mixture (final concentration of 0.2 μM and 0.1 μM, respectively), and 2.5 μL of 4× TaqPath™ 1-Step RT-qPCR Master Mix (Catalog number A28526, Thermo Fisher Waltham, Mass.) or 4× inhibitor-Tolerant RT-qPCR mix (MDX016-50, Meridian Bioscience, Tennessee). Thermal cycling was performed at 25° C. for 2 min for uracil-N-glycosylase gene (UNG) incubation and 53° C. for 10 mm for reverse transcription, followed by 95° C. for 2 min and then 45 cycles of 95° C. for 3 sec, and 60° C. for 30 sec. QuantStudio™ 5 Real-Time PCR System (Thermo Fisher, USA) were used for rRT-PCR amplification and detection.

### Statistical Data Analysis

Average cycle threshold (Ct), standard deviation (SD) and coefficient of variation (CV) were calculated using Microsoft Office Excel 365 software (Microsoft, Redmond, Wash.). Clinical so sensitivity, specificity, positive percent agreement (PPA) and negative percent agreement (NPA) at two-sided 95% confidence interval (CI) were analyzed using MedCalc software Version 19.3.1

### Results

### Analytical Sensitivity

Non-infectious viral particles from the AccuPlex SARS-CoV-2 Reference Material Kit (SeraCare Bioscience) were spiked in saliva at various concentrations (50, 100 and 200 copies/mL). Real-time RT-PCR assay was performed with the provided kit reagents. The assessment of individual assay result is that sample Ct<40 indicates positive and Ct>40 indicates negative. Therefore, 100 copies/mil, were determined as a tentative LOD due to 50 copies/mL, sample was undetectable (Table 21).

TABLE 21
Tentative LOD determination by series dilution*

| | viral RNA | | | |
|---|---|---|---|---|
| Target | (copy/mL) | Avg Ct | SD | CV |
| ORF1ab | 50 | 39.1 | 3.3 | 8% |
| | 100 | 33.4 | 0.7 | 2% |
| | 200 | 33.1 | 0.6 | 2% |
| N gene | 50 | 37.2 | 0.2 | 1% |
| | 100 | 33.7 | 1.1 | 3% |
| | 200 | 32.6 | 0.4 | 1% |
| E gene | 50 | 40.1 | 3.6 | 9% |
| | 100 | 35.8 | 0.1 | 0% |
| | 200 | 35.2 | 0.2 | 1% |
| Rp gene | 50 | 31.9 | 0.4 | 1% |
| | 100 | 31.4 | 0.1 | 0% |
| | 200 | 31.8 | 0.2 | 1% |

*For each individual RT-PCR assay, a Ct value < 40 indicates positive and a Ct > 40 indicates negative. Accordingly, 100 copies/mL were determined as the tentative LOD.

We then validated the QuantiVirus™ SARS-CoV-2 kit on four qPCR instruments from different vendors, using contrived saliva samples by 20 measurements. The overall analytical sensitivity (lower limit of detection or LOD) is around 100-200 copies/mL under 95% confidence interval (Table 2). The validation data established that the LOD of the assay is 200 copies/mL on ABI 7500 Fast Dx (Table 22a), 100 copies/mL on Bio-Rad CFX384 (Table 22b), 200 copies/mL on Roche LightCycler 480 II (Table 22c), and 200 copies/mL on the Thermo Fisher QuantStudio 5 (Table 22d).

TABLE 22a
Summary of twenty replicates for analytical sensitivity confirmation on the ABI 7500 Dx

| Target | RNA (copy/mL) | Total | Avg Ct | SD | CV | Positive | Negative | Call Rate |
|---|---|---|---|---|---|---|---|---|
| ORF1ab gene | 100 copies/mL | 20 | 34.28 | 1.05 | 3.08% | 20 | 0 | 100% |
| N gene | 100 copies/mL | 20 | 35.73 | 1.12 | 3.13% | 20 | 0 | 100% |
| E gene | 200 copies/mL | 20 | 34.24 | 0.98 | 2.87% | 20 | 0 | 100% |

TABLE 22b
Summary of twenty replicates for analytical sensitivity confirmation on the BioRad CFX 384

| Target | RNA (copy/mL) | Total | Avg Ct | SD | CV | Positive | Negative | Call Rate |
|---|---|---|---|---|---|---|---|---|
| ORF1ab gene | 100 copies/mL | 20 | 33.76 | 0.97 | 2.87% | 20 | 0 | 100% |
| N gene | 100 copies/mL | 20 | 35.97 | 1.02 | 2.85% | 20 | 0 | 100% |
| E gene | 100 copies/mL | 20 | 37.87 | 0.58 | 1.52% | 20 | 0 | 100% |

TABLE 22c
Summary of twenty replicates for analytical sensitivity confirmation on the Roche LC 480

| Target | RNA (copy/mL) | Total | Avg Ct | SD | CV | Positive | Negative | Call Rate |
|---|---|---|---|---|---|---|---|---|
| ORF1ab gene | 100 copies/mL | 20 | 32.85 | 0.57 | 1.7% | 20 | 0 | 100% |
| N gene | 200 copies/mL | 20 | 35.04 | 0.58 | 1.7% | 20 | 0 | 100% |
| E gene | 100 copies/mL | 20 | 36.13 | 0.59 | 1.6% | 20 | 0 | 100% |

TABLE 22d
Summary of twenty replicates for analytical sensitivity confirmation on the ABI QS5

| Target | RNA (copy/mL) | Total | Avg Ct | SD | CV | Positive | Negative | Call Rate |
|---|---|---|---|---|---|---|---|---|
| ORF1ab gene | 200 copies/mL | 20 | 34.09 | 0.66 | 1.92% | 20 | 0 | 100% |
| N gene | 200 copies/mL | 20 | 35.11 | 1.81 | 5.14% | 20 | 0 | 100% |
| E gene | 200 copies/mL | 20 | 34.99 | 1.68 | 4.82% | 20 | 0 | 100% |

### Clinical Evaluation

Saliva samples were collected and tested with QuantiVirus™ SARS-CoV-2 Kit. A set of patient saliva samples with known status was tested with the QuantiVirus™ SARS-CoV-2 Test using the ABI QuantStudio 5. Total 40 saliva positive samples and 40 negative samples were tested. Data indicated 100% sensitivity and 100% specificity for saliva samples (Tables 23 and 24).

TABLE 23
Saliva Positive Sample Detected by QuantiVirus ™ SARS-CoV-2 Test

| | DiaCarta QuantiVirus SARS-CoV-2 4plex assay | | | | |
|---|---|---|---|---|---|
| | FAM (ORF | TEX | Cy5 | HEX | |
| Sample | 1ab gene) | (E gene) | (N gene) | (RP gene) | Result |
| Positive Saliva Sample 1 | 27.329 | 28.597 | 30.931 | 23.543 | SARS-CoV-2 detected |
| Positive Saliva Sample 2 | 29.773 | 30.232 | 31.971 | 23.142 | SARS-CoV-2 detected |
| Positive Saliva Sample 3 | 24.728 | 26.038 | 27.375 | 22.722 | SARS-CoV-2 detected |
| Positive Saliva Sample 4 | 28.001 | 28.395 | 30.478 | 25.121 | SARS-CoV-2 detected |
| Positive Saliva Sample 5 | 14.998 | 15.617 | 17.062 | 23.075 | SARS-CoV-2 detected |
| Positive Saliva Sample 6 | 24.173 | 24.644 | 26.551 | 23.331 | SARS-CoV-2 detected |
| Positive Saliva Sample 7 | 27.616 | 28.120 | 30.672 | 24.006 | SARS-CoV-2 detected |
| Positive Saliva Sample 8 | 20.244 | 21.192 | 23.218 | 23.614 | SARS-CoV-2 detected |
| Positive Saliva Sample 9 | 22.378 | 23.462 | 25.037 | 23.120 | SARS-CoV-2 detected |
| Positive Saliva Sample 10 | 29.217 | 29.025 | 30.697 | 22.742 | SARS-CoV-2 detected |
| Positive Saliva Sample 11 | 28.021 | 28.595 | 30.203 | 24.297 | SARS-CoV-2 detected |
| Positive Saliva Sample 12 | 26.063 | 27.002 | 27.663 | 25.019 | SARS-CoV-2 detected |
| Positive Saliva Sample 13 | 20.406 | 21.948 | 23.268 | 22.237 | SARS-CoV-2 detected |
| Positive Saliva Sample 14 | 26.934 | 27.395 | 29.041 | 23.447 | SARS-CoV-2 detected |
| Positive Saliva Sample 15 | 33.833 | 32.844 | 39.897 | 23.264 | SARS-CoV-2 detected |
| Positive Saliva Sample 16 | 25.045 | 25.212 | 27.046 | 21.629 | SARS-CoV-2 detected |
| Positive Saliva Sample 17 | 25.731 | 26.325 | 27.895 | 24.136 | SARS-CoV-2 detected |
| Positive Saliva Sample 18 | 21.102 | 22.294 | 23.788 | 23.457 | SARS-CoV-2 detected |
| Positive Saliva Sample 19 | 19.666 | 21.041 | 22.442 | 23.963 | SARS-CoV-2 detected |
| Positive Saliva Sample 20 | 31.532 | 31.812 | 34.705 | 21.276 | SARS-CoV-2 detected |
| Positive Saliva Sample 21 | 33.078 | 31.574 | 34.978 | 24.876 | SARS-CoV-2 detected |
| Positive Saliva Sample 22 | 29.830 | 30.163 | 32.058 | 27.611 | SARS-CoV-2 detected |
| Positive Saliva Sample 23 | 33.335 | 31.490 | 34.315 | 22.881 | SARS-CoV-2 detected |
| Positive Saliva Sample 24 | 31.323 | 31.126 | 33.051 | 20.614 | SARS-CoV-2 detected |
| Positive Saliva Sample 25 | 16.618 | 17.619 | 19.607 | 23.445 | SARS-CoV-2 detected |
| Positive Saliva Sample 26 | 28.798 | 29.365 | 31.692 | 24.591 | SARS-CoV-2 detected |
| Positive Saliva Sample 27 | 26.969 | 27.784 | 29.901 | 22.820 | SARS-CoV-2 detected |
| Positive Saliva Sample 28 | 20.141 | 21.270 | 22.838 | 24.095 | SARS-CoV-2 detected |
| Positive Saliva Sample 29 | 30.278 | 30.571 | 32.596 | 24.099 | SARS-CoV-2 detected |
| Positive Saliva Sample 30 | 32.289 | 30.984 | 33.319 | 24.613 | SARS-CoV-2 detected |
| Positive Saliva Sample 31 | 14.649 | 15.187 | 16.806 | 22.548 | SARS-CoV-2 detected |
| Positive Saliva Sample 32 | 24.725 | 25.899 | 27.385 | 22.914 | SARS-CoV-2 detected |
| Positive Saliva Sample 33 | 28.495 | 28.826 | 30.198 | 24.645 | SARS-CoV-2 detected |
| Positive Saliva Sample 34 | 18.666 | 19.999 | 21.857 | 22.627 | SARS-CoV-2 detected |
| Positive Saliva Sample 35 | 29.583 | 29.783 | 32.007 | 24.418 | SARS-CoV-2 detected |
| Positive Saliva Sample 36 | 17.402 | 18.415 | 18.677 | 21.517 | SARS-CoV-2 detected |
| Positive Saliva Sample 37 | 28.294 | 28.558 | 30.759 | 24.005 | SARS-CoV-2 detected |
| Positive Saliva Sample 38 | 17.008 | 18.151 | 19.381 | 23.809 | SARS-CoV-2 detected |
| Positive Saliva Sample 39 | 23.330 | 24.072 | 25.550 | 25.470 | SARS-CoV-2 detected |
| Positive Saliva Sample 40 | 34.244 | 35.326 | 35.258 | 28.052 | SARS-CoV-2 detected |

TABLE 24
Saliva Negative Sample Detected by QuantiVirus ™ SARS-CoV-2 Test

| | FAM(ORF | TEX | Cy5 | HEX | |
|---|---|---|---|---|---|
| Sample | lab gene) | (E gene) | (N gene) | (RP′gene) | Result |
| Negative Saliva Sample 1 | Undetermined | Undetermined | Undetermined | 24.513 | SARS-CoV- 2 Not Detected |
| Negative Saliva Sample 2 | Undetermined | Undetermined | Undetermined | 23.176 | SARS-CoV- 2 Not Detected |
| Negative Saliva Sample 3 | Undetermined | Undetermined | Undetermined | 26.197 | SARS-CoV- 2 Not Detected |
| Negative Saliva Sample 4 | Undetermined | Undetermined | Undetermined | 23.875 | SARS-CoV- 2 Not Detected |
| Negative Saliva Sample 5 | Undetermined | Undetermined | Undetermined | 25.213 | SARS-CoV- 2 Not Detected |
| Negative Saliva Sample 6 | Undetermined | Undetermined | Undetermined | 23.922 | SARS-CoV- 2 Not Detected |
| Negative Saliva Sample 7 | Undetermined | Undetermined | Undetermined | 25.235 | SARS-CoV- 2 Not Detected |
| Negative Saliva Sample 8 | Undetermined | Undetermined | Undetermined | 23.590 | SARS-CoV- 2 Not Detected |
| Negative Saliva Sample 9 | Undetermined | Undetermined | Undetermined | 24.035 | SARS-CoV- 2 Not Detected |
| Negative Saliva Sample 10 | Undetermined | Undetermined | Undetermined | 24.216 | SARS-CoV- 2 Not Detected |
| Negative Saliva Sample 11 | Undetermined | Undetermined | Undetermined | 23.164 | SARS-CoV- 2 Not Detected |
| Negative Saliva Sample 12 | Undetermined | Undetermined | Undetermined | 23.754 | SARS-CoV- 2 Not Detected |
| Negative Saliva Sample 13 | Undetermined | Undetermined | Undetermined | 22.432 | SARS-CoV- 2 Not Detected |
| Negative Saliva Sample 14 | Undetermined | Undetermined | Undetermined | 24.564 | SARS-CoV- 2 Not Detected |
| Negative Saliva Sample 15 | Undetermined | Undetermined | Undetermined | 25.036 | SARS-CoV- 2 Not Detected |
| Negative Saliva Sample 16 | Undetermined | Undetermined | Undetermined | 25.305 | SARS-CoV- 2 Not Detected |
| Negative Saliva Sample 17 | Undetermined | Undetermined | Undetermined | 23.957 | SARS-CoV- 2 Not Detected |
| Negative Saliva Sample 18 | Undetermined | Undetermined | Undetermined | 25.043 | SARS-CoV- 2 Not Detected |
| Negative Saliva Sample 19 | Undetermined | Undetermined | Undetermined | 24.726 | SARS-CoV- 2 Not Detected |
| Negative Saliva Sample 20 | Undetermined | Undetermined | Undetermined | 23.209 | SARS-CoV- 2 Not Detected |
| Negative Saliva Sample 21 | Undetermined | Undetermined | Undetermined | 21.466 | SARS-CoV- 2 Not Detected |
| Negative Saliva Sample 22 | Undetermined | Undetermined | Undetermined | 23.122 | SARS-CoV- 2 Not Detected |
| Negative Saliva Sample 23 | Undetermined | Undetermined | Undetermined | 21.793 | SARS-CoV- 2 Not Detected |
| Negative Saliva Sample 24 | Undetermined | Undetermined | Undetermined | 21.947 | SARS-CoV- 2 Not Detected |
| Negative Saliva Sample 25 | Undetermined | Undetermined | Undetermined | 25.192 | SARS-CoV- 2 Not Detected |
| Negative Saliva Sample 26 | Undetermined | Undetermined | Undetermined | 24.280 | SARS-CoV- 2 Not Detected |
| Negative Saliva Sample 27 | Undetermined | Undetermined | Undetermined | 22.588 | SARS-CoV- 2 Not Detected |
| Negative Saliva Sample 28 | Undetermined | Undetermined | Undetermined | 22.975 | SARS-CoV- 2 Not Detected |
| Negative Saliva Sample 29 | Undetermined | Undetermined | Undetermined | 24.302 | SARS-CoV- 2 Not Detected |
| Negative Saliva Sample 30 | Undetermined | Undetermined | Undetermined | 24.145 | SARS-CoV- 2 Not Detected |
| Negative Saliva Sample 31 | Undetermined | Undetermined | Undetermined | 24.001 | SARS-CoV- 2 Not Detected |
| Negative Saliva Sample 32 | Undetermined | Undetermined | Undetermined | 23.352 | SARS-CoV- 2 Not Detected |
| Negative Saliva Sample 33 | Undetermined | Undetermined | Undetermined | 28.934 | SARS-CoV- 2 Not Detected |
| Negative Saliva Sample 34 | Undetermined | Undetermined | Undetermined | 23.685 | SARS-CoV- 2 Not Detected |
| Negative Saliva Sample 35 | Undetermined | Undetermined | Undetermined | 24.790 | SARS-CoV- 2 Not Detected |
| Negative Saliva Sample 36 | Undetermined | Undetermined | Undetermined | 27.876 | SARS-CoV- 2 Not Detected |
| Negative Saliva Sample 37 | Undetermined | Undetermined | Undetermined | 26.094 | SARS-CoV- 2 Not Detected |
| Negative Saliva Sample 38 | Undetermined | Undetermined | Undetermined | 24.360 | SARS-CoV- 2 Not Detected |
| Negative Saliva Sample 39 | Undetermined | Undetermined | Undetermined | 23.773 | SARS-CoV- 2 Not Detected |
| Negative Saliva Sample 40 | Undetermined | Undetermined | Undetermined | 22.055 | SARS-CoV- 2 Not Detected |

Summary, using the QuantiVirus™ SARS-CoV-2 Test, we tested clinical saliva samples with known status including 40 positive samples and 40 negative samples (Table 25). The data show that the positive percent value (PPA) is 100% (95% CI: 0.891 to 1.00) and negative percent value (NPA) is 100% (95% CI: 0.891 to 1.00).

TABLE 25
Clinical Saliva Samples Evaluation with ABIQS5 qPCR Instrument
by the QuantiVirus ™ SARS-CoV-2 Test

| | | QuantiVirus | | | |
|---|---|---|---|---|---|
| Patient | | SARS-CoV-2 Test | | PPA | NPA |
| Samples | N | Detected | Not detected | (95% CI) | (95% CI) |
| Positive | 40 | 40 | 0 | 100% | 100% |
| Negative | 40 | 0 | 40 | (0.891-1.00) | (0.891-1.0) |

Applicant also conducted a comparison of the instant invention (QuantiVirus™ SARS-CoV-2 multiplex kit with FDA EUA approved) Abbott Realtime SARS-CoV-2 kit, We tested 24 saliva samples of recovering COVID-19 patients with the QuantiVirus™ SARS-CoV-2 kit in comparison with the Abbott m2000 RealTime SARS-CoV-2 PCR kit in parallel (Table 26). Data showed a concordance of the assays of about 88%. There were three samples detected by QuantiVirus™ SARS-CoV-2 kit, hut not detectable with the Abbott kit (patients #8, 11 and 12), consistent with the reported higher sensitivity of QuantiVirus™ SARS-COV-2 PCR assay.

TABLE 26
Comparison of Abbott m2000 SARS-CoV-2 PCR test and DiaCarta QuantiVirus ™ SARS-
CoV-2 PCR test for SARS-CoV-2 detection in clinical saliva samples.

| Method | Abbott m2000 Real-time SARS-CoV-2 | | Diacarta QuantiVirus SARS-CoV-2 multiplex | | | | |
|---|---|---|---|---|---|---|---|
| Comparison | Accession # | Detection & qPCR Ct | Detection | ROME-Gene) | Cy5(N-Gene) | FAM(ORF lab gene) | VIC(RP Gene) |
| Patient 1 | Saliva 1 | Not Detected | Not Detected | Undetermined | Undetermined | Undetermined | 21.6 |
| Patient 2 | Saliva 2 | Not Detected | Not Detected | Undetermined | undetermined | Undetermined | 22.9 |
| Patient 3 | Saliva 3 | Not Detected | Not Detected | Undetermined | undetermined | Undetermined | 22.1 |
| Patient 4 | Saliva 4 | Not Detected | Not Detected | Undetermined | undetermined | Undetermined | 21.6 |
| Patient 5 | Saliva 5 | Detected (Cs 18.21) | Detected | 32.1 | 31.7 | 30 | 20.7 |
| Patient 6 | Saliva 6 | Detected (Ct 31.00) | Detected | Undetermined | 37.8 | Undetermined | 23.7 |
| Patient 7 | Saliva 7 | Not Detected | Not Detected | Undetermined | Undetermined | Undetermined | 19.7 |
| Patient 8 | Saliva 8 | Not Detected | Detected | 37.4 | 37.3 | 42.4 | 23.3 |
| Patient 9 | Saliva 9 | Detected (Ct 23.89). | Detected | Undetermined | 24.4 | undetermined | 21.8 |
| Patient 10 | Saliva 10 | Not Detected | Not Detected | Undetermined | undetermined | Undetermined | 22.3 |
| Patient 11 | Saliva 11 | not detected | Detected | 32.8 | 33.9 | 31.5 | 23.9 |
| Patient 12 | Saliva 12 | not detected | Detected | 35.7 | 38.5 | 33.9 | 24.2 |
| Patient 13 | Saliva 13 | Not Detected | Not detected | Undetermined | 43.5 | Undetermined | 24.5 |
| Patient 14 | Saliva 14 | Detected (Ct 20.77) | Detected | 36.1 | 37.0 | 34 | 26.1 |
| Patient 15 | Saliva 15 | Detected | Detected | 35.6 | 35.1 | 34.2 | 23.6 |
| Patient 16 | Saliva 16 | Not Detected. | Not detected | Undetermined | 37.7 | Undetermined | 23.6 |
| Patient 17 | Saliva 17 | Not Detected | Not detected | Undetermined | Undetermined | 41.2 | 32.7 |
| Patient 18 | Saliva 18 | Not Detected | Not detected | Undetermined | Undetermined | Undetermined | 24.9 |
| Patient 19 | Saliva 19 | Not Detected | Not detected | Undetermined | Undetermined | 33.5 | 29.1 |
| Patient 20 | Saliva 20 | Detected (Ct. 21.40) | Detected | Undetermined | 39.4 | 36.8 | 26.6 |
| Patient 21 | Saliva 21 | Not Detected | Not detected | Undetermined | Undetermined | Undetermined | 25.1 |
| Patient 22 | Saliva 22 | Not Detected | Not detected | Undetermined | 43.5 | Undetermined | 23.6 |
| Patient 23 | Saliva 23 | Not Detected | Not detected | Undetermined | Undetermined | Undetermined | 29.1 |
| Patient 24 | Saliva 24 | Not Detected | Not detected | Undetermined | Undetermined | Undetermined | 25.2 |

We also tested 389 total saliva specimens collected from the general population of asymptomatic individuals (ie, asymptomatic screening) in Los Angeles and the San Francisco Bay Area counties. The screened population was represented by African Americans, White, Asian, and Latinx, with ages ranging from 18 to 80 (average 41 years old. From May 8 to Aug. 26, 2020, 301 saliva samples were tested, and 5 samples were tested positive for BARS-CoV-2 by the QuantiVirus™ SARS-CoV-2 test. The 5 positives corresponded to 4 males of ages 19, 51, 52 and 54, and 1 female of age 34. Overall detection rate was 1.66% (Table 27). In another testing run of 88 saliva. samples, 2 samples were positive and 86 were negative, with an overall positive detection rate of 2.27%. Together, we had screened 389 people from the general population and found that 7 people were positive for SARS-CoV-2 with an overall detection rate of 1.8%, consistent with the reported. average positive testing rate from the same periods in the two metropolitan regions.

TABLE 27
Summary of saliva-based COVID-19 screening using
QuantiVirus ™ SARS-CoV-2 test in local communities

| | Total | | | Detection |
|---|---|---|---|---|
| Date | (N) | Positive | Negative | Rate (%) |
| May 8-Aug. 26, 2020 | 301 | 5 | 296 | 1.66% |
| Aug. 28, 2020 | 88 | 2 | 86 | 2.27% |
| Total | 389 | 7 | 382 | 1.80% |

We also tested the feasibility of pooling saliva specimens for screening asymptomatic patients, we pooled negative and positive saliva samples, and tested a total of 77 pooled positive samples (1 patient sample mixed with 5 healthy saliva samples; 1:6 ratio) and 54 pooled negative samples (mixed 6 healthy samples) (Table 28). Of the 77 pooled positive saliva samples, 73 were tested positive (average Ct of three genes: O gene Ct˜29.8; E gene 30.9 and N gene Ct˜31.0) and 4 was reported as undetected. The average internal control (IC) RP Ct was 21.9 for all 131 pooled samples. Positive Predictive Value (PPV) is 100% (95% CI: 93.8%-100%). Negative Predictive Value (NPV) is 93.1% (95% CI: 82.5-97.8%). Additionally, we tested a total of 49 pooled positive saliva samples, created by mixing 1 patient sample with 11 healthy samples (1:12 ratio). Of the 49 pooled positive samples, 44 were tested positive (0 (gene, E gene and N gene average Ct 31.8, 32.1 and 31.9) and 5 was reported as undetected. Its IC RP average Ct was 22.3 for all 49 pooled saliva. samples and additional 20 pooled healthy saliva samples. PPV is 100% (95% CI: 89.9%-100%) and NPV is 80.0% (95% CI: 58.7%-92.4%), respectively.

TABLE 28
Saliva sample pooling for SARS-CoV-2 detection by QuantiVirus ™ SARS-COV-2 test kit.

| Saliva Sample | Sample | | | Total Screen | | | | |
|---|---|---|---|---|---|---|---|---|
| Pooling | Test (N) | Positive | Negative | Sample(N) | Sensitivity | Specificity | PPV (%) | NPV (%) |
| 1 positive + 5 | 77 | 73 | 4 | 462 | 94.8% | 100% | 100% | 93.1% |
| negative pooling | | | | | (95% CI: | (95% CI: | (95% CI: | (95% CI: |
| 6 negative pooling | 54 | 0 | 54 | 324 | 0.865-0.983) | 0.917-1.00) | 0.938-1.00) | 0.825-0.978) |
| 1 positive + 11 | 49 | 44 | 5 | 588 | 89.8% | 100% | 100% | 80.0% |
| negative pooling | | | | | (95% CI: | (95% CI | (95% CI: | (95% CI: |
| 12 negative pooling | 20 | 0 | 20 | 240 | 0.769-0.962) | 0.799-1.00) | 0.899-1.00) | 0.587-0.924) |

### Example III

### Methods

### Study Design and Ethics

Besides contrived saliva samples, deidentified leftover patient NPS and saliva samples were used in the study. All patient specimens were collected in May-September 2020 and previously tested at UCSF affiliated San Francisco VAMC clinical laboratories and DiaCarta's CLIA laboratory for clinical diagnostic or screening purpose. Other than qualitative RT-PCR results (positive or negative), only PCR cycle threshold (Ct) values were included in study analysis and no patient clinical chart reviews were performed. This study was approved by the institutional review board (IRB) at UCSF (UCSF IRB 411-05207) as a no-subject contact study with waiver of consent and as exempt under category 4,

### Clinical Specimens

Clinical samples were collected from patients who had previously been tested positive for SARS-CoV-2. Paired NPS and saliva samples were collected at the same time. The QuantiVirus™ Saliva Collection Kit (DiaCarta, Inc. cat #DC-11-0021) was used for saliva collection, following the kit insert instructions and under the supervision of healthcare providers. No eat or drink 30 minutes before saliva sample collection,

Each saliva sample contains about 2 mL liquid saliva and 2 mL viral transport media. The NPS and saliva samples are refrigerated and processed for testing within 24 hours after collection.

### Sample Pooling

Positive saliva and negative saliva samples were pooled together according to the experiment design for 1:6 (i.e., 1 positive mixed with 5 negatives) and 1:12 (i.e., 1 positive mixed with 11 negatives) pooling, respectively. A total of 77 positive patient samples and 385 negative samples were used for pooling at 1:6 ratio to create 77 pooled positive samples and 54 pooled negative samples. After mixing the pooled samples, RNA was extracted for RT-PCR according to the testing protocol.

### Viral RNA Extraction

MGI's automatic RNA/DNA extraction instrument MGISP-960 (MGI Tech Co., Ltd, China) was used for the SARS-CoV-2 viral RNA extraction according to the manufacturer's instructions, for which 200 μL of each NPS VIM or saliva sample was used. For each batch of clinical samples to be tested, an extraction control (EC) was included (spike 20 μL of EC from the QuantiVirus™ SARS-CoV-2 kit into 180 μL sterile RNase-free water). The clinical samples and spiked EC were processed and extracted on the MGI platform. The extraction output is RNA in 30-50 μL RNase-free water, 5.5 μL of which is used for the PCR reaction per test. The turnaround time from sample extraction to PCR final report is around 4 hrs (FIG. 1B). Precautions were taken while handling extracted RNA samples to avoid RNA degradation. Extracted RNA samples were stored at −80° C. if not immediately used for RT-PCR.

### Multiplex Primer and Probe Design

Target gene sequences in the SARS-CoV-2 genome, the N gene, E gene and ORF1ab gene were identified and selected for test development. The gene sequences were retrieved from GenBank. and GISAID databases for primer and probe designs to ensure coverage of all SARS-CoV-2 strains. Multiple alignments of the collected sequences were performed using Qiagen CLC Main Workbench 20.0.4., and conserved regions in each target gene were identified using BioEditor 7.2.5. prior to primer and probe designs. Primers and probes were designed to target the most conserved regions of each of the target genes of the viral genome, using Primer3plus software and following general rules of real-time PCR design. All primers were designed with a melting temperature (Tm) of approximately 60° C. and the probes were designed. with a Tm of about 65° C. The amplicon sizes were kept as short as possible within the range of 70 by to 150 by for each primer pair to achieve better amplification efficiency and detection sensitivity. All primers and probes were synthesized by Integrated DNA Technologies, Inc. IDT, Coralville, Iowa, USA) and LGC Biosearch Technologies (Novato, Calif., USA), respectively.

### Real-Time Reverse-Transcription PCR (RRT-PCR)

The total volume of one RT-PCR reaction for all targets is 10 μL, including 5.5 μL of RNA, 2.0 μL of 5× primer and probe mixture (final concentration of 0.2 and 0.1 μM, respectively), and 2.5 μL, of 4×TaqPath™ 1-Step RT-qPCR Master Mix (Catalog number A28526, Thermo Fisher, Waltham, Mass.) or 4× Inhibitor-Tolerant RT-qPCR mix (MDX016-50, Meridian Bioscience, Tennessee). Thermal cycling was performed at 25° C. for 2 min for uracil-N-glycosylase gene (UNG) incubation and 53° C. for 10 min for reverse transcription, followed by 95° C. for 2 min and then 45 cycles of 95° C. for 3 sec, and 60° C. for 30 sec. QuantStudio™ 5 Real-Time PCR System (Thermo Fisher, USA), Applied Biosystems™ 7500 Fast Dx Real-Time PCR Instrument (Thermo Fisher, USA), BioRad CFX384 (Bio-Rad, USA) and Roche LightCycler 480 II (Roche, USA) were used for rRT-PCR amplification and detection.

### Analytical Sensitivity and Limit of Detection (LoD)

To determine the Limit of Detection (LoD) and analytical sensitivity of the Quanti Virus SARS CoV-2 Test kit, studies using empirical method were performed using serial dilutions of analyte and the LoD was determined to be the lowest concentration of template that could reliably be detected with 95% of all tested positive. LoD of each target assay in the QuantiVirus™ SARS-CoV-2 Test were conducted and verified using SeraCare AccuPlex SARS-CoV-2 Reference Material Kit (Cat #0505-0126). Non-infectious viral particles from the AccuPlex SARS-CoV-2 Reference Material Kit were spiked in saliva at various concentrations (50 copies/ml, 100 copies/mL, and 200 copies/mL) diluted from the stock concentration of 5000 copies/mL. Real-time RT-PCR assay was performed with the provided kit reagents and tested triplicate on ABI QS5, ABI 7500 Fast Dx, Bio-Rad CFX 384 PCR and Roche LightCycler 480 II instruments. Then the LOD was confirmed by testing 1×LoD of viral RNA with 20 replicates. The was determined to be the lowest concentration (copies/ml) at which >95% (19/20) of the 20 replicates were tested as positive.

### Precision

Precision studies include intra-run, inter-run, instrument, and operator variability evaluation. The assay precision was assessed by the repeat testing of samples with three or more different template concentrations. (1) Inter-assay % CV was established for same lot of reagents tested on the same instrument by the same user; (2) Intra-assay % CV was established through performance of kit on reference samples run in replicates of nine; and (3) Operator variability was evaluated with one lot of reagents by two operators. Reproducibility is demonstrated based on % CV of Ct values.

### Microorganism Panel for Cross-Reactivity

MERS-coronavirus, SARS-CoV coronavirus samples were ordered from IDT. NATtrol Respiratory Validation Panel was ordered from ZeptoMetrix (cat #NATRVP-3, Buffalo, N.Y.). RNA/DNA were extracted from high titer stocks of the potentially cross-reacting microorganisms.

### Statistical Data Analysis

Average cycle threshold (Ct), standard deviation (SD) and coefficient of variation (CV) were calculated using Microsoft Office Excel 365 software (Microsoft, Redmond, Wash.). Clinical sensitivity, specificity, positive predictive value (PPV) and negative predictive value (NPV) at two-sided 95% confidence interval (CI) were analyzed using MedCalc software Version 19.3.1, NP and saliva pair analysis was conducted by Wilcoxon signed rank test.

### Results

### Validation of QuantiVirus™ SARS-Cov-2 Test Kit

### Analytical Sensitivity

Non-infectious viral particles from the AccuPlex SARS-CoV-2 Reference Material Kit (SeraCare Bioscience) were spiked in saliva at various concentrations (50, 100 and 200 copies/mL). RT-PCR assay was performed with the provided kit reagents. The assessment of individual assay result is that sample Ct<40 indicates positive and Ct>40 indicates negative. Therefore, 100 copies/mL were determined as a tentative LOD due to 50 copies/mL sample was undetectable from E gene target.

We then validated the QuantiVirus SARS-CoV-2 kit on four qPCR instruments from different vendors, using contrived saliva samples by 20 measurements. The overall analytical sensitivity (lower limit of detection or LOD) is around 100-200 copies/mL under 95% confidence interval.

The validated multiplex rRT-PCR assay of the invention for SARS-CoV-2 detection in saliva samples with clinical sensitivity of 98.8% (95% CI: 92.7%-99.9%) and specificity of 100% (95% CI: 94.9%100%). Its PPV is 100% (95% CI: 94.6%-100%) and NPV is 98.9% (95% CI: 93.1%-99.9%). The detection of three viral target genes in one PCR tube enables a high throughput test using RT-qPCR. For these validated 384-well plate PCR platforms, 381 patient samples can be tested in each run (plus 3 controls). We have validated and integrated MGISP-960 high-throughput Automated Sample Preparation System, which can extract 192 samples (2×96) in about 80 min. For a CLIA laboratory with two MGI-960 machines, 380 samples can be tested with results available within 4 hrs.

We spiked SARS-CoV-2 viral particles into healthy donor saliva and confirmed that the analytical sensitivity (LOD) of the QuantiVirus™ RT-qPCR test is ˜100 copies/mL for Bio-Rad CFX 384 and ˜200 copies/mL fix ABI QS5 ABI 7500Dx and Roche LC 480. Comparing to other FDA approved test kits, we have confirmed that our test kit has 600 NAAT Detectable Units/mL (NDU/mL) by FDA Reference Panel Testing and is among the top of all FDA approved SARS-CoV-2 test kits. The multiplex. RT-qPCR test can simultaneously detect three viral gene targets, which can minimize false negative results as Chances of simultaneous mutations in all three target genes in the viral genome are highly unlikely. Furthermore, the results confirm that human saliva samples do not inhibit the RT-qPCR reaction, possibly due to the fact that inhibitor-tolerant RT-PCR. master mix was used in the QuantiVirus™ SARS-CoV-2 test kit.

The SARS-CoV-2 test results were 87.5% in concordance with FDA EUA approved Abbott RealTime SARS-CoV-2 results for saliva samples, with a higher detection rate overall. In fact, this observation is consistent with recently reported test sensitivity among various SARS-CoV-2 molecular tests. FDA published its SARS-CoV-2 Reference Panel Comparative Data on its website on Sep. 15, 2020. It reported that QuantiVirus™ BARS-CoV-2 Kit has LOD of 600 NDU/mL whereas Abbott Realtime SARS-CoV-2 assay has LOD of 2700 NDU/mL. Accordingly, the reason for the observation that SARS-CoV-2 viral RNA was detected in three patient samples by the QuantiVirus™ SARS-CoV-2 test but not by Abbott RealTime SARS-COV-2 assay was likely due to the higher sensitivity of the QuantiVirus™ SARS-CoV-2 assay. it also demonstrated that saliva specimens represent a viable specimen type that can be easily applied for COVID-19 testing when using more sensitive tests.

A total of 389 saliva specimens from the general population were tested and demonstrated the feasibility of using saliva for large scale population screening. Saliva is a non-invasive and easily collectable specimen for COVID-19 screening. Given the drawbacks of nasopharyngeal and oropharyngeal swab sample collection, saliva sampling could be applied as an acceptable alternative.

With saliva pooling strategy, we have demonstrated that 6-samples pooling (1 patient mixed with 5 healthy saliva samples, or 1:6 ratio) has 94.8% sensitivity (95% CI: 86.5-983%) and 100% specificity (95% CI:91.7-100%), As noted, of the 77 pooled saliva samples, 4 pooling samples were tested negative. In fact, for these 4 pooled samples, the individual. positive samples used for the pooling had Ct of 34.4, 34.8, 35.7 and 37.5 for ORFlab gene, respectively, consistent with low viral loads to start with (less than 100-200 copies/mL) (see Table 1a-1d). Therefore, in order to detect weakly positive patient in pooled samples, a RT-PCR test with LOD at 100-200 copies/mL or higher is required. If pooling testing is considered, each clinical laboratory should establish laboratory-specific pooling protocol based on the LOD of SARS-CoV-2 molecular test. One advantage of pooling testing is its cost-effectiveness, allowing population-based asymptomatic screening or monitoring even when. testing supplies are limited.

In summary, we have demonstrated that saliva specimens can be reliably used for SANS-CoV-2 detection, and saliva-based large-scale population screening for COVID-19 with or without pooling is feasible.

### REFERENCES

1. Zhu N, Zhang D, Wang W, Li X, Yang B, Song J, et al. A Novel Coronavirus from Patients with Pneumonia in China, 2019. N Engl J Med. 2020 Jan. 24.
2. World Health Organization (WHO). Coronavirus. Geneva: WHO; 2020 [Accessed 21 Jan. 2020].
3. Li Q, Guan X, Wu P, Wang X, Zhou L, Tong Y, et al. Early Transmission Dynamics in Wuhan, China, of Novel Coronavirus-Infected Pneumonia. N Engl J Med. 2020 Jan. 29.
4. Chen N, Zhou M, Dong X, Qu J, Gong F, Han Y, et al. Epidemiological and clinical characteristics of 99 cases of 2019 novel coronavirus pneumonia in Wuhan, China: a descriptive study. Lancet. 2020 Jan. 30.
5. Chan J F, Yuan S, Kok K H, To K K, Chu H, Yang J, et al. A familial cluster of pneumonia associated with the 2019 novel coronavirus indicating person-to-person transmission: a study of a family cluster. Lancet. 2020 Jan. 24
6. Holshue M L, DeBolt C, Lindquist S, Lofy K H, Wiesman J, Bruce H, et al. First Case of 2019 Novel Coronavirus in the United States. N Engl J Med. 2020 Jan. 31
7. Zhou, P. et al. A pneumonia outbreak associated with a new coronavirus of probable bat origin. Nature. 2020
8. Wu, F. et al. A new coronavirus associated with human respiratory disease in China. Nature, doi:10.1038/s41586-020-2008-3 (2020).
9 Lu, R. et al. Genomic characterization and epidemiology of 2019 novel coronavirus: implications for virus origins and receptor binding. Lancet, doi:10.1016/S0140-6736(20)30251-8 (2020).
10. Carmon et al. Detection of 2019 novel coronavirus (2019-nCoV) by real-time RT-PCR. Euro Surveill. 2020; 25(3):2000045
11. Lucia et al. An ultrasensitive, rapid, and portable coronavirus SARS-CoV-2 sequence detection 3 method based on CRISPR-Cas12. BioRxiV 2020: 1-10

All literature and similar materials cited in this application including, but not limited to, patents, patent applications, articles, books, treatises, and internet web pages, regardless of the format of such literature and similar materials, are expressly incorporated by reference in their entirety for any purpose as if they were entirely denoted. In the event that one or more of the incorporated literature and similar materials defines or uses a term in such a way that it contradicts that term's definition in this application, this application controls.

Although the foregoing description contains many specifics, these should not be construed as limiting the scope of the present invention, but merely as providing illustrations of some of the presently preferred embodiments. Similarly, other embodiments may be devised without departing from the spirit or scope of the present invention. Features from different embodiments may be employed in combination. The scope of the invention is, therefore, indicated and limited only by the appended claims and their legal equivalents rather than by the foregoing description. All additions, deletions and modifications to the invention as disclosed herein which fall within the meaning and scope of the claims are to be embraced thereby.

What is claimed is:

1. A PCR primer set useful for detecting SARS-CoV-2 selected from the group consisting of the following primer sets: (a) a primer set comprising a primer consisting of WHnCoVF2 SEQ ID NO: 1 GTTCCAATTAACA CCAATAGCA and a primer WHnCoVR2a SEQ ID NO: 2 ATTCGTCTGGTAGCTCTTC (b) a primer set comprising a primer consisting of WHnCoVF3 SEQ ID NO: 4 GCAAATTCTATGGTGGTTGG and a primer consisting of WHnCoVR3 SEQ ID NO: 5 GCATGGCTCTATCACATTTAG (c) a primer set comprising a primer consisting of WHnCoVF4 SEQ ID NO: 7 GCTTCGATTGTGTGCGTAC and a primer consisting of WHnCoVR4 SEQ ID NO: 8 GACCAGAAGATCAGGAACTCTA; and (d) a primer set comprising a primer consisting of RP-FSEQ ID NO: 10 AGATTTGGACCTGCGAGCG and a primer consisting of RP-R SEQ ID NO: 11 GAGCGGCTGTCTCCACAAGT; wherein the primer set specifically amplifies a target region of Severe Acute Respiratory syndrome corona virus CoV-2 (SARS-CoV-2) in a polymerase chain reaction (PCR).

2. Oligonucleotides, for use as a probe to detect the amplified nucleic acid sequence resulting in the amplification of a target sequence located within the genome of SARS Coronavirus-2, said amplification being based on pair of oligonucleotides according to claim 1, said probe being selected from the group consisting of WHnCoVPr2 (Probe) SEQ ID NO: 3 TCCAGATGACCAAATTGGCTAC; WHnCoVPr3(Probe) SEQ ID NO: 6 ACTGTTTATAGT GATGTAGAAAACCCTCA; WHnCoVPr4(Probe) SEQ ID NO: 9 CTGCAATATTGTTAACG TGAGTCTTGT; and RP-P (Probe) SEQ ID NO: 12 TTCTGACCTGAA GGCTCTGCGCG.

3. A method for determining the presence or absence of SARS-associated corona virus Cov-2 (SARS-CoV-2) in a biological sample, the method comprising: (a) contacting nucleic acid from a biological sample with at least one primer which is a nucleic acid of claim 1, (b) subjecting the nucleic acid and the primer to amplification conditions, and (c) determining the presence or absence of amplification product, wherein the presence of amplification product indicates the presence of RNA associated with corona virus in the sample.

4. A method for detecting SARS-associated corona virus Cov-2 (SARS-CoV-2) by contacting a biological sample with a set of primers and a probe, incubating under conditions allowing amplification of nucleic acid using said primers, and determining binding of said probe to amplified nucleic acid, wherein detecting binding of said probe to amplified nucleic acid indicates the presence of SARS-associated virus, wherein the primers are selected from the group consisting of the following primer sets: (a) a primer set comprising a primer consisting of WHnCoVF2 SEQ ID NO: 1 GTTCCAATTAACACCAATAGCA and a primer WHnCoVR2a SEQ ID NO: 2 ATTCGTCTGGTAGCTCTTC; (b) a primer set comprising a primer consisting of WHnCoVF3 SEQ ID NO: 4 GCAAATTCTATGGTGGTTGG and a primer consisting of WHnCoVR3 SEQ ID NO: 5 GCATGGCTCTATCACATTTAG; (c) a primer set comprising a primer consisting of WHnCoVF4 SEQ ID NO: 7 GCTTCGATTGTGTGCGTAC and a primer consisting of WHnCoVR4 SEQ ID NO: 8 GACCAGAAGATCAGGAACTCTA; and (d) a primer set comprising a primer consisting of RP-FSEQ ID NO: 10 AGATTTGGACC TGCGAGCG and a primer consisting of RP-R SEQ ID NO: 11 GAGCGGCTGTCTCCACAAG T; and wherein the probe is selected from the group consisting of WHnCoVPr2 (Probe) SEQ ID NO: 3 TCCAGATGACCAAATTGGCTAC; WHnCoVPr3 (Probe) SEQ ID NO: 6 ACTGTTTATAGT GATGTAGAAAACCCTCA; WHnCoVPr4(Probe) SEQ ID NO: 9 CTGCAATATTGTT AACGTGAGTCTTGT; and RP-P (Probe) SEQ ID NO: 12 TTCTGACCTGAAGGCTCTGC GCG; and wherein the probe is labeled with two dyes, one dye of which is a fluorescent reporter dye, and one dye of which is a quencher dye, and wherein at least one dye is a fluorescent dye; and the SARS virus is detected by detection of real time fluorescence, if amplification of virus specific sequence occurs.

5. The method of claim 4, wherein the amplification and detection are performed using real time RT-PCR.

6. Method according to claim 4, wherein the primer set are WHnCoVF2 SEQ ID NO: 1 GTTCCAATTAACACCAATAGCA and WHnCoVR2a SEQ ID NO: 2 ATTCGTCTGGTAGC TCTTC and wherein the probe has the sequence shown as WHnCoVPr2 (Probe) SEQ ID NO: 3 TCCAGATGACCAAATTGGCTAC.

7. Method according to claim 4, wherein the reporter dye is FAM, 6-FAM, 5-FAM and ALEXA-288.

8. Method according to claim 4, wherein the quencher dye is TAMRA, DABCYL or QSY.

9. Method according to claim 4, wherein detection is quantitative detection of the real time fluorescence signal intensity.

10. Method according to claim 4, wherein the biological sample is a body fluid.

11. Method according to claim 10, wherein the body fluid is sputum, saliva, nasopharyngeal fluid, oropharyngeal fluid or blood.

12. Kit for detecting SARS-associated corona virus Cov-2 (SARS-CoV-2) in a biological sample comprising a PCR primer set selected from the group consisting of the following primer sets: (a) a primer set comprising a primer consisting of WHnCoVF2 SEQ ID NO: 1 GTTCCAATTAACA CCAATAGCA and a primer WHnCoVR2a SEQ ID NO: 2 ATTCGTCTGGTAGCTCTTC (b) a primer set comprising a primer consisting of WHnCoVF3 SEQ ID NO: 4 GCAAATTCTATGGTGGTTGG and a primer consisting of WHnCoVR3 SEQ NO: 5 GCATGGCTCTATCACATTTAG; (c) a primer set comprising a primer consisting of WHnCoVF4 SEQ ID NO: 7 GCTTCGATTGTGTGCGTAC and a primer consisting of WHnCoVR4 SEQ ID NO: 8 GACCAGAAGATCAGGAACTCTA; and (d) a primer set comprising a primer consisting of RP-FSEQ ID NO: 10 AGATTTGGACCTGCGAGCG and a primer consisting of RP-R SEQ ID NO: 11 GAGCGGCTGTCTCCACAAGT; wherein the primer set specifically amplifies a target region of Severe Acute Respiratory syndrome corona virus CoV-2 (SARS-CoV-2) in a polymerase chain reaction (PCR).

13. Kit according to claim 12, wherein the reporter dye is FAM, 6-FAM, 5-FAM and ALEXA-288.

14. Kit according to claim 12, wherein the quencher dye is TAMRA, DABCYL or QSY.

15. Kit according to claim 12, further comprising enzymes and reagents required for performing a real time RT-PCR reaction.


SEQUENCE LISTING (16 sequences)

SEQ ID NO: 1 - DNA, 22 bp, Artificial
  Synthetic
gttccaattaacaccaatagca

SEQ ID NO: 2 - DNA, 19 bp, Artificial
  Synthetic
attcgtctggtagctcttc

SEQ ID NO: 3 - DNA, 22 bp, Artificial
  Synthetic
tccagatgaccaaattggctac

SEQ ID NO: 4 - DNA, 20 bp, Artificial
  Synthetic
gcaaattctatggtggttgg

SEQ ID NO: 5 - DNA, 21 bp, Artificial
  Synthetic
gcatggctctatcacatttag

SEQ ID NO: 6 - DNA, 29 bp, Artificial
  Synthetic
actgtttatagtgatgtagaaaaccctca

SEQ ID NO: 7 - DNA, 19 bp, Artificial
  Synthetic
gcttcgattgtgtgcgtac

SEQ ID NO: 8 - DNA, 22 bp, Artificial
  Synthetic
gaccagaagatcaggaactcta

SEQ ID NO: 9 - DNA, 27 bp, Artificial
  Synthetic
ctgcaatattgttaacgtgagtcttgt

SEQ ID NO: 10 - DNA, 19 bp, Artificial
  Synthetic
agatttggacctgcgagcg

SEQ ID NO: 11 - DNA, 20 bp, Artificial
  Synthetic
gagcggctgtctccacaagt

SEQ ID NO: 12 - DNA, 23 bp, Artificial
  Synthetic
ttctgacctgaaggctctgcgcg

SEQ ID NO: 13 - DNA, 200 bp, Artificial
  Synthetic
gacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgca

SEQ ID NO: 14 - DNA, 200 bp, Artificial
  Synthetic
accgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctgg

SEQ ID NO: 15 - DNA, 270 bp, Artificial
  Synthetic
accgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgt

SEQ ID NO: 16 - DNA, 336 bp, Artificial
  Synthetic
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaat